(19) United States
(12) Patent Application Publication
(10) Pub. No.: US 2007/0088051 A1
(43) Pub. Date: Apr. 19, 2007

(54) INDOLINONES AND THEIR USE AS ANTIPROLIFERATIVE AGENTS

(75) Inventors: Darryl McConnell, Vienna (AT); Ulrike Weyer-Czernilofsky, Baden (AT); Maria Impagnatiello, Vienna (AT); Steffen Steurer, Vienna (AT); Ralph Brueckner, Vienna (AT); Bernd Krist, Vienna (AT); Bodo Betzemeier, Vienna (AT); Frank Hilberg, Vienna (AT); Armin Heckel, Biberach (DE); Gerald Juergen Roth, Biberach (DE); Joerg Kley, Mittelbiberach (DE); Thorsten Lehmann-Lintz, Ochsenhausen (DE)

Correspondence Address: MICHAEL P. MORRIS;BOEHRINGER INGELHEIM CORPORATION, 900 RIDGEBURY RD, P. O. BOX 368, RIDGEFIELD, CT, 06877-0368, US

(73) Assignee: Boehringer Ingelheim International GmbH, Ingelheim (DE)

(21) Appl. No.: 11/558,953

(22) Filed: Nov. 13, 2006

Related U.S. Application Data
(63) Continuation of application No. 11/303,168, filed on Dec. 16, 2005, now abandoned.

(30) Foreign Application Priority Data
Dec. 17, 2004 (EP) 04029981

(51) Int. Cl. A61K 31/4709; A61K 31/4439; A61K 31/421; A61K 31/423; A61K 31/422; A61K 31/416; A61K 31/404; C07D 413/02; C07D 403/02

(52) U.S. Cl. 514/314; 514/337; 514/374; 514/375; 514/406; 514/414; 548/465; 546/169; 546/277.7; 548/215; 548/216; 548/364.7; 514/418

(57) ABSTRACT

The invention relates to indolinone compounds of formula (I), 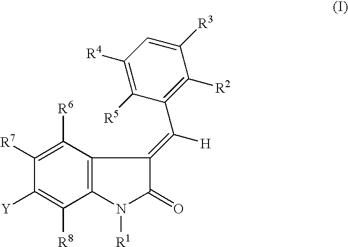
wherein Y and R¹ to R⁸ are defined as in claim 1, which are suitable for the treatment of diseases characterized by excessive or abnormal cell proliferation and the use thereof for preparing a pharmaceutical composition.

### SUMMARY OF THE INVENTION

The invention relates to indolinone compounds of formula (I), 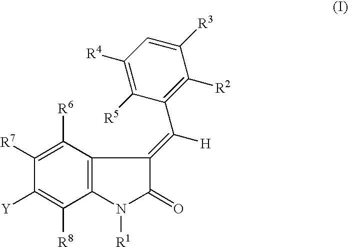
to their tautomers, enantiomers, diastereomers, mixtures thereof and their physiologically acceptable salts which have useful pharmacological properties and which possess tubulin inhibitory activity and their use as antiproliferative agents.

### BACKGROUND OF THE INVENTION

Microtubules are cytoskeletal structures assembled from α/β tubulin heterodimers that play an essential role in many cellular processes, such as cell motility, organelle transport, maintenance of cell polarity and cell division. Interference with microtubule dynamics by stabilization or destabilization in dividing cells leads to cell division arrest in the G₂/M phase and cell death.

A variety of clinically promising compounds which demonstrate potent cytotoxicity and antitumor activity are known to effect their primary mode of action through an efficient inhibition of tubulin. Several natural products and their derivatives disrupt microtubule dynamics, e.g., Taxol®, Taxotere®, Navelbine® and show a clinically useful therapeutic window between anticancer effects and dose-limiting toxicity in normal proliferating tissues, notably bone marrow and gastrointestinal mucosa in addition to neurotoxicity.

Unfortunately the clinical success of these agents can be severely hindered by the emergence of drug resistant tumor cells. Although membrane P-glycoprotein mediated multi-drug resistance (MDR) has been known to occur with the taxanes and the Vinca alkaloids, differential expression of altered tubulin isotypes has also been implicated in resistance to the taxanes and other antimitotic agents. Renewed interest in tubulin polymerisation inhibitors has been generated by the hope that non-MDR substrates that interact with tubulin at sites near to, overlapping with or different from those of the taxanes or the Vinca alkaloids can be discovered.

Novel tubulin-binding molecules which, upon binding to tubulin, interfere with tubulin polymerization can provide novel agents for the treatment of proliferative diseases.

WO9640116 discloses and claims indolinone-derivatives bearing an alkoxy, aryloxy, hydroxy or halogen substituent either in ortho-position or in para-position of the benzylidenyl moiety as tyrosine kinase activity modulators. WO9807695 describes combinatorial libraries and related products for the treatment of cell proliferative diseases and metabolic diseases. Indolinones bearing a heteroaryl in position 6 are described in WO0056709.

### DETAILED DESCRIPTION OF THE INVENTION

Surprisingly it was found that compounds of formula (I), wherein the residues R¹-R⁸ and Y have the meaning as defined herein, can act as tubulin polymerisation inhibitors.

The invention therefore relates to a compound of formula (I) 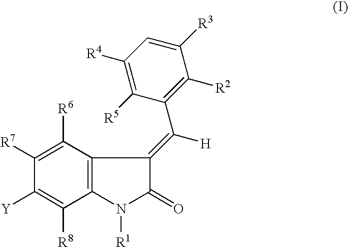
to their tautomers, enantiomers, diastereomers, mixtures thereof and their physiologically acceptable salts thereof, wherein

R¹ is H or methyl; and

R², R³, R⁴ and R⁵ are independently selected from the group consisting of hydrogen, cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNₐOR′ₐ, —(CH₂)ₓONRₐR′ₐ, —(CH₂)ₓOC(═O)NR′ₐRₐ, —(CH₂)ₓNRₐC(═O)OR′ₐ, (CH₂)ₓOC(═O)Rₐ, —(CH₂)ₓC(═O)ORₐ, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ, —(CH₂)ₓNR″ₐC(═O)NRₐR′ₐ and —[(CH₂)ₓO—]_{y}R_{g} or from an optionally substituted group consisting of C₁₋₆alkyl, biaryl, carbocyclic aryl, heteroalicyclo and heteroaryl; and

R⁶, R⁷ and R⁸ are independently selected from the group consisting of hydrogen, hydroxy, thiol, halo, cyano, amino, methylamino, dimethylamino, nitro and CF₃ or from an optionally substituted group selected from C₁₋₄alkoxy, C₁₋₄alkylthio, C₁₋₆alkyl, wherein the substituents are selected from the group consisting of halo, hydroxy and oxo; and

Y is selected from the group consisting of cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ and —(CH₂)ₓNR″ₐC(═O)NRₐR′ₐ or from an optionally substituted group consisting of C₁₋₆alkyl, biaryl, carbocyclic aryl, heteroalicyclo and heteroaryl; and

R² and R³, R⁴ and R⁵ and R⁷ and Y may also combine to form a cycloalkyl, cycloalkenyl, cycloalkynyl, carbocyclic aryl, heteroalicyclo or heteroaryl ring; and

x is an integer selected from 0, 1, or 2; and

y is an integer selected from 1, 2 or 3; and

Rₐ, R′ₐ and R″ₐ are independently selected from hydrogen or from an optionally substituted group consisting of C₁₋₆alkyl, cycloalkyl, heteroalicyclo and aryl; wherein optionally Rₐ and R′ₐ, Rₐ and R″ₐ and R′ₐ and R″ₐ, may combine to form a heteroalicyclic ring; and

R_{d} is selected from hydrogen or from an optionally substituted group consisting of amino, C₁₋₆alkyl, cycloalkyl, heteroalicyclo, carbocyclic aryl, heteroaryl, C₁₋₄alkoxy, aryloxy, N-amido, N-thioamido and urea; and

Rₑ is selected from the group consisting of hydrogen and hydroxy or from an optionally substituted group consisting of C₁₋₆alkyl, C₁₋₄alkoxy, aryloxy, cycloalkyl, heteroalicyclo, carbocyclic aryl and heterocyclic aryl; and

R_{f} is selected from the group consisting of hydrogen and cyano or from an optionally substituted group consisting of C₁₋₆alkyl, cycloalkyl, heteroalicyclo, carbocyclic aryl and heterocyclic aryl; and

R_{g} is selected from the group consisting of hydrogen and C₁₋₆alkyl.

A further aspect of the invention is a compound of formula (I), wherein R², R³, R⁴ and R⁵ are independently selected from the group consisting of hydrogen, cyano, hydroxy, halo, nitro, thiol, —(CH₂)ₓC(═O)Rₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓ(Rₐ)C═NR_{d}, —(CH₂)ₓS(═O)₂Rₐ, —(CH₂)ₓS(═O)Rₐ, —(CH₂)ₓSRₐ, —(CH₂)ₓS(═O)₂Rₐ, —(CH₂)ₓOS(═O)₂Rₐ, —(CH₂)ₓNR″ₐS(═O)₂NRₐR′ₐ, —(CH₂)ₓS(═O)₂NRₐR′ₐ and —(CH₂)ₓNRₐS(═O)₂R′ₐ or from an optionally substituted group consisting of C₁₋₆alkyl, C₁₋₆alkenyl, C₁₋₆alkynyl, carbocyclic aryl, heteroalicyclo and heteroaryl.

An additional aspect of the invention is a compound of formula (I), wherein R² is selected from the group consisting of hydrogen, hydroxy, halo, —(CH₂)ₓC(═O)NRₐR′ₐ, —(CH₂)ₓNRₐC(═O)R′ₐ, —(CH₂)ₓNRₐR′ₐ and —(CH₂)ₓORₐ or from an optionally substituted group consisting of carbocyclic aryl, heteroalicyclo and heteroaryl.

Another aspect of the invention is a compound of formula (I), wherein

R³ is selected from the group consisting of hydrogen, hydroxy, halo, —(CH₂)ₓC(═O)NRₐR′ₐ, —(CH₂)ₓNRₐC(═O)R′ₐ, —(CH₂)ₓNRₐR′ₐ or —(CH₂)ₓORₐ or from an optionally substituted group consisting of carbocyclic aryl, heteroalicyclo and heteroaryl.

Yet, another aspect of the invention is a compound of formula (I), wherein R² is selected from the group consisting of hydrogen, hydroxy, amino and halo.

One aspect of the invention is a compound of formula (I), wherein R³ is selected from the group consisting of hydrogen, hydroxy, amino and halo.

Another aspect of the invention is a compound of formula (I), wherein R⁴ is selected from the group consisting of hydrogen, hydroxy, amino and halo.

A further aspect of the invention is a compound of formula (I), wherein R⁵ is elected from the group consisting of hydrogen, hydroxy, amino and halo.

An alternative aspect of the invention is a compound of formula (I), wherein Y is selected from the group consisting of hydroxy, halo, thiol, —(CH₂)ₓC(═O)Rₐ, —(CH₂)ₓC(═O)NRₐR′ₐ, —CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓ(Rₐ)C═NR_{d}, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ and —(CH₂)ₓNRₐS(═O)₂R′ₐ or from an optionally substituted group consisting of C₁₋₆alkyl, C₁₋₆alkenyl, C₁₋₆alkynyl, carbocyclic aryl, heteroalicyclo and heteroaryl.

An aspect of the invention is a compound of formula (I), wherein Y is selected from the group consisting of hydroxy, —(CH₂)ₓNRₐR′ₐ, —(CH₂)ₓOC(═O)Rₐ, —(CH₂)ₓC(═O)ORₐ or —(CH₂)ₓORₐ or from an optionally substituted group consisting of carbocyclic aryl, heteroalicyclo and heteroaryl.

A further aspect of the invention is a compound of formula (I), wherein Y is selected from the group consisting of bromo, hydroxy, methoxy, ethoxy, allyloxy, isopropoxy, carboxy, methoxycarbonyl, ethoxycarbonyl, propoxycarbonyl, methylcarbamoyl, ethylcarbamoyl, benzyl-methyl-carbamoyl, oxazol, benzooxazol, furanyl, pyrrolyl, pyrazolyl, thiophenyl, phenyl, cyano-phenyl, methoxy-phenyl, acetylaminophenyl, benzodioxolyl, pyridinyl, methyl-pyridinyl and quinolinyl.

An additional aspect of the invention is a compound of formula (I), wherein R⁶, R⁷ and R⁸ are independently selected from the group consisting of hydrogen, hydroxy, halo, cyano, amino, methylamino, dimethylamino, methyl and CF₃.

One aspect of the invention is a compound of formula (I), wherein R¹ is hydrogen.

A further aspect of the invention is a compound of formula (I) as medicament.

Another aspect of the invention is a compound of formula (I) as antiproliferative medicament.

Yet, another aspect of the invention is the use of a compound of formula (I) for the manufacture of a medicament for the treatment of a proliferative disease.

Also an aspect of the invention is the use of a compound of formula (I) for the manufacture of a medicament for the treatment of cancer.

An alternative aspect of the invention is the use of a compound of formula (I) for the manufacture of a medicament for the treatment of conditions ameliorated by an inhibitory action on tubulin polymerization

A further aspect of the invention is a pharmaceutical composition containing as active ingredient one or more compounds of formula (I), or their physiologically acceptable salts, in combination with a usual adjuvants and/or carrier.

Another aspect of the invention is a pharmaceutical composition comprising a compound of formula (I) 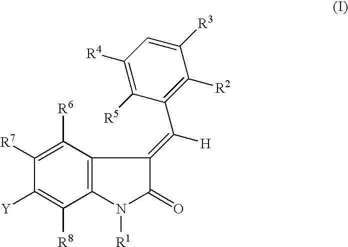
wherein

R¹ is H or methyl; and

R², R³, R⁴ and R⁵ are independently selected from the group consisting of hydrogen, cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ—, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ, —(CH₂)ₓNR″ₐC(═O)NRₐR′ₐ and —[(CH₂)ₓO—]_{y}R_{g} or from an optionally substituted group consisting of C₁₋₆alkyl, biaryl, carbocyclic aryl, heteroalicyclo and heteroaryl; and

R⁶, R⁷ and R⁸ are independently selected from the group consisting of hydrogen, hydroxy, thiol, halo, cyano, amino, methylamino, dimethylamino, nitro and CF₃ or from an optionally substituted group selected from C₁₋₄alkoxy, C₁₋₄alkylthio and C₁₋₆alkyl, wherein the substituents are selected from the group consisting of halo, hydroxy and oxo; and

Y is selected from the group consisting of cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ and —(CH₂)ₓNR″ₐC(═O)NRₐR′ₐ or from an optionally substituted group consisting of C₁₋₆alkyl, biaryl, carbocyclic aryl, heteroalicyclo and heteroaryl; and

R² and R³, R⁴ and R⁵ and R⁷ and Y may also combine to form a cycloalkyl, cycloalkenyl, cycloalkynyl, carbocyclic aryl, heteroalicyclo or heteroaryl ring; and

x is an integer selected from 0, 1, or 2; and

y is an integer selected from 1, 2 or 3; and

Rₐ, R′ₐ and R″ₐ are independently selected from hydrogen or from an optionally substituted group consisting of C₁₋₆alkyl, cycloalkyl, heteroalicyclo and aryl; wherein optionally Rₐand R′ₐ, Rₐ and R″ₐ and R′ₐ and R″ₐ, may combine to form a heteroalicyclic ring; and R_{d} is selected from hydrogen or from an optionally substituted group consisting of amino, C₁₋₆alkyl, cycloalkyl, heteroalicyclo, carbocyclic aryl, heteroaryl, C₁₋₄alkoxy, aryloxy, N-amido, N-thioamido and urea; and

Rₑ is selected from the group consisting of hydrogen and hydroxy or from an optionally substituted group consisting of C₁₋₆alkyl, C₁₋₄alkoxy, aryloxy, cycloalkyl, heteroalicyclo, carbocyclic aryl and heterocyclic aryl; and

R_{f} is selected from the group consisting of hydrogen and cyano or from an optionally substituted group consisting of C₁₋₆alkyl, cycloalkyl, heteroalicyclo, carbocyclic aryl and heterocyclic aryl; and

R_{g} is selected from the group consisting of hydrogen and C₁₋₆alkyl;

or a tautomers, enantiomer, diastereomer, mixture thereof, pharmaceutically acceptable salt, solvate or physiologically functional derivative thereof, and

at least one different cytostatic and/or cytotoxic active ingredient or a pharmaceutically acceptable salt, solvate, or physiologically functional derivative thereof, and a pharmaceutically acceptable carrier or excipient.

An additional aspect of the invention is the use of a compound of formula I and at least one different cytostatic and/or cytotoxic active ingredient or a pharmaceutically acceptable salt, solvate, or physiologically functional derivative thereof in a combined form, or separately or separately and sequentially, wherein the sequential administration is close in time or remote in time, for the manufacture of a medicament for the prevention or treatment of a proliferative disease.

Definitions

As used herein, the following definitions shall apply unless otherwise indicated.

The term “optionally substituted” is used interchangeably with the phrase “substituted or unsubstituted” and means that a group may be substituted by one or more substituents which may be the same or different. When otherwise not specified these substituents are selected from alkyl, cycloalkyl, biaryl, carbocyclic aryl, heteroalicyclo, heteroaryl, acyl, amidino, amido, amino, alkoxyamino, carbamoyl, carboxy, cyano, ether, guanidine, hydroxamoyl, hydroxyl, imino, isocyanato, isothiocyanato, halo, nitro, silyl, sulfonyl, sulfinyl, sulfenyl, sulfonato, sulfamoyl, sulfonamido, thiocarbonyl, thiol, thiocyanato, thiocarbamoyl, thioamido or urea as those terms are define herein.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond. An “alkyne” moiety refers to a group consisting of at least two carbon atoms and at least one carbon-carbon triple bond. The alkyl moiety, whether saturated or unsaturated, may be branched or non-branched. Branched means that the alkyl moiety is substituted by one or more lower alkyl groups such as for example methyl, ethyl or propyl

The alkyl group may have the number of carbon atoms as explicitly defined (e.g. C₁₋₁₂alkyl) or may also be undefined. Whenever it appears herein a numerical range such as “1 to 12” it refers to each integer in the given range. For example, “1 to 12 carbon atoms” means that the alkyl group may consist of 1 carbon atom, 2 carbon atoms, 3 carbon atoms, etc., up to and including 12 carbon atoms. When the number of carbon atoms is undefined the alkyl group has 1 to 12 carbon atoms. A medium sized alkyl refers to an alkyl group having 1 to 8 carbon atoms. A lower alkyl group refers to an alkyl group having 1 to 5 carbon atoms. The alkyl group, whether termed an alkyl, saturated alkyl, unsaturated alkyl, alkene or alkyne, may be unsubstituted or substituted as defined herein.

The term “carbocyclic” refers to a compound which contains one or more covalently closed ring structures and the atoms forming the backbone of the ring(s) are all carbon atoms. The term thus distinguishes carbocyclic from “heterocyclic” rings. Carbocyclic groups include both, a “cycloalkyl” group, which means a non-aromatic carbocycle, and a “carbocyclic aryl” group, which means an aromatic carbocycle. The carbocyclic group may be optionally substituted as defined herein.

The term “cycloalkyl” as used herein refers to mono-, bi- or tricyclic rings or ring systems. The ring system may be a “saturated ring”, which means that the ring does not contain any alkene or alkyne moieties. The cycloalkyl group may also be an “unsaturated ring” which means that it contains at least one alkene or alkyne moiety and provided that the ring system is not aromatic. The cycloalkyl group may be unsubstituted or substituted as defined herein and the substituents, when positioned adjacent to one another, may combine to form carbocyclic or heterocyclic ring systems for example methylendioxy or difluoromethylendioxy. In addition to the above mentioned substituents one or more ring carbon atoms may also be bonded via a double bond to a heteroatom selected from N, S and O and wherein N may optionally be substituted by Rₐ.

The term “aryl” as used herein refers to a mono-, bi- or tricyclic ring or ring systems which have at least one aromatic ring. Aryl groups include both, “carbocyclic aryl” and “heteroaryl” groups. The aryl moiety may be unsubstituted or substituted as defined herein and the substituents, when positioned adjacent to one another, may combine to form cycloalkyl or heteroalicyclic ring systems for example methylendioxy or difluoromethylendioxy.

The term “biaryl” as used herein refers to two aryl groups, as defined herein, joined together via a single bond. The biaryl moiety may be unsubstituted or substituted as defined herein and the substituents, when positioned adjacent to one another, may combine to form cycloalkyl or heteroalicyclic ring systems for example methylendioxy or difluoromethylendioxy.

The term “carbocyclic aryl” as used herein refers to mono-, bi- or tricyclic rings or ring systems which have at least one aromatic ring and all atoms forming the backbone are carbon atoms. Examples of carbocyclic aryl groups include but are not limited to phenyl, naphthyl and anthracyl. The carbocyclic aryl moiety may be unsubstituted or substituted as defined herein and the substituents, when positioned adjacent to one another, may combine to form cycloalkyl or heteroalicyclic ring systems for example methylendioxy or difluoromethylendioxy.

The term “heterocyclic” or “heterocyclo” as used herein refers to mono-, bi- or tricyclic rings or ring systems which include one or more heteroatoms selected from N, S and O. The rings or ring systems include 1 to 13 carbon atoms in addition to the heteroatom(s). The term heterocyclic group include both, a “heteroalicyclic” group, which means a non-aromatic heterocycle and a “heteroaryl” group, which means an aromatic heterocycle. The heterocyclic moiety may be unsubstituted or substituted as defined herein and the substituents, when positioned adjacent to one another, may combine to form cycloalkyl or heteroalicyclic ring systems for example methylendioxy or difluoromethylendioxy. The heterocyclic group may be bonded via a carbon atom or a heteroatom. The heterocyclic group may also include the oxides of nitrogen and sulfur if nitrogen or sulfur are present in the ring.

The term “heteroalicyclic” or “heteroalicyclo” as used herein refers to mono-, bi- or tricyclic ring or ring systems in which at least one of the atoms forming the backbone of the ring is a heteroatom. The ring system may be a “saturated ring”, which means that the ring does not contain any alkene or alkyne moieties, or it may also be an “unsaturated ring” which means that it contains at least one alkene or alkyne moiety provided that the ring system is not aromatic. The heteroalicyclic group may be unsubstituted or substituted as defined herein. The substituents, when positioned adjacent to one another, may combine to form carbocyclic or heterocyclic ring systems for example methylendioxy or difluoromethylendioxy. The heteroalicyclic group may be bonded via a carbon atom or a heteroatom. In addition to the above mentioned substituents one or more ring carbon atoms may also be bonded via a double bond to a heteroatom selected from N, S and O and wherein N may optionally be substituted by R₃. The heteroalicyclic group may also include the oxides of nitrogen and sulfur if nitrogen or sulfur are present in the ring.

The term “heteroaryl”, “heterocyclic aryl” or “heteroaromatic radical” as used herein refers to a mono-, bi- or tricyclic rings or ring systems which include one or more heteroatoms selected from N, S and O. The rings or ring systems include 1 to 13 carbon atoms in addition to the heteroatom(s) and contains at least one aromatic ring with a heteroatom. The heteroaryl group may also include the oxides of nitrogen and sulfur if nitrogen or sulfur are present, respectively. Examples of monocyclic heteroaryl groups include but are not limited to furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl and triazinyl. Examples of bicyclic heterocycles include but are not limited to indolyl, benzofuranyl, benzothienyl, benzoxazolyl, benzothiazolyl, benzisoxazolyl, benzisothiazolyl, indazolyl, isoquinolinyl, quinolinyl, quinoxalinyl, cinnolinyl, phthalazinyl, quinazolinyl, benzotriazinyl and the like. Examples of tricyclic heterocycles include but are not limited to thianthrenyl, xanthenyl, phenoxathiinyl, carbazolyl, carbolinyl, phenanthridinyl, acridinyl, perimidinyl, phenanthrolinyl, phenazinyl, phenothiazinyl, phenoxazinyl. The heteroaryl group may be unsubstituted or substituted as defined herein. The substituents, when positioned adjacent to one another, may combine to form a cycloalkyl or heteroalicyclic ring for example methylendioxy and difluoromethylendioxy. The heteroaryl radical may be bonded via a carbon atom or a heteroatom.

The term “heteroarylalkyl”, as used herein, refers to a chemical moiety of formula heteroaryl-(CH₂)ₓ— as those terms are defined herein.

The term “carbocyclic arylalkyl”, as used herein, refers to a chemical moiety of formula carbocyclic aryl-(CH₂)ₓ— as those terms are defined herein.

The term “biarylalkyl”, as used herein, refers to a chemical moiety of formula biaryl-(CH₂)ₓ— as those terms are defined herein.

The term “heteroarylalkyl”, as used herein, refers to a chemical moiety of formula heteroaryl-(CH₂)ₓ— as those terms are defined herein.

The term “heteroalicycloalkyl”, as used herein, refers to a chemical moiety of formula heteroalicyclo-(CH₂)ₓ— as those terms are defined herein.

The term “cycloalkylalkyl”, as used herein, refers to a chemical moiety of formula cycloalkyl-(CH₂)ₓ— as those terms are defined herein.

The term “acyl”, as used herein, refers to a chemical moiety of formula —(CH₂)ₓC(═O)Rₐ.

The term “amidino” refers to a chemical moiety with the formula —(CH₂)ₓC(═NH)NRₐR′ₐ.

The term “amido” refers to both, a “C-amido” group which means a chemical moiety with the formula —(CH₂)ₓC(═O)NRₐR′ₐ and a “N-amido” group which means a chemical moiety with the formula —(CH₂)ₓNRₐC(═O)R′ₐ.

The term “amine” or “amino” refers to a chemical moiety of formula —(CH₂)ₓNRₐR′ₐ. The definition of an amine is also understood to include their N-oxides.

The term “alkoxyamino”, refers to both, an “N-alkoxyamino” group which means a chemical moiety with the formula —(CH₂)ₓNRₐOR′ₐ and an “O-alkoxyamino” group which means a chemical moiety with the formula —(CH₂)ₓONRₐR′ₐ.

The term “carbamoyl” refers to both, an “O-carbamoyl” group which means a chemical moiety with the formula —(CH₂)ₓOC(═O)NRₐR′ₐ and a “N-carbamoyl” group which means a chemical moiety with the formula —(CH₂)ₓNRₐC(═O)OR′ₐ.

The term “carboxy” refers to both, an “O-carboxy” group which means a chemical moiety with the formula —(CH₂)ₓOC(═O)Rₐ and a “C-carboxy” group which means a chemical moiety with the formula —(CH₂)ₓC(═O)ORₐ.

A “cyano” group refers to a —(CH₂)ₓC≡N.

The term “ether” refers to a chemical moiety of formula —(CH₂)ₓORₐ.

The term “guanidino” refers to a chemical moiety with the formula —(CH₂)ₓNHC(═NH)NHR_{f}.

The term “hydroxamoyl” refers to a chemical moiety with the formula —(CH₂)ₓC(═O)NORₐ.

The term “hydroxy” or “hydroxyl” as used herein, refers to a chemical moiety of formula —OH.

The term “imine” or “imino”, as used herein, refers to a chemical moiety of formula —(CH₂)ₓ(Rₐ)C═NR_{d}.

An “isocyanato” group refers to a —NCO group.

An “isothiocyanato” group refers to a —NCS group.

The term “halogen” or “halo” refers to an atom selected from the group consisting of fluorine, chlorine, bromine and iodine.

The term “silyl”, as used herein, refers to to a chemical moiety with the formula —Si(Rₑ)₃.

The term “sulfone” or “sulfonyl” refers to a chemical moiety with the formula —(CH₂)ₓS(═O)₂Rₐ.

The term “sulfinyl” refers to a chemical moiety with the formula —(CH₂)ₓS(═O)Rₐ.

The term “sulfenyl” refers to a chemical moiety with the formula —(CH₂)ₓSRₐ.

The term “sulfonato” refers to both, an “S-sulfonato” group which means a chemical moiety with the formula —(CH₂)ₓS(═O)₂ORₐ and an “O-sulfonato” group which means a chemical moiety with the formula —(CH₂)ₓOS(═O)₂Rₐ.

A “sulfamoyl” group refers to a chemical moiety with the formula —(CH₂)ₓNR″ₐS(═O)₂NRₐR′ₐ.

The term “sulfonamido” refers to both, an “S-sulfonamido” group which means a chemical moiety with the formula —(CH₂)ₓS(═O)₂NRₐR′ₐ and an “N-sulfonamido” group which means a chemical moiety with the formula —(CH₂)ₓNRₐS(═O)₂R′ₐ.

The term “thiocarbonyl” refers to a chemical moiety with the formula —(CH₂)ₓC(═S)R′ₐ.

The term “thio” or “thiol”, as used herein, refers to a chemical moiety of formula —SH.

A “thiocyanato” group refers to a —CNS group.

The term “thiocarbamoyl” refers to both, an “O-thiocarbamoyl” group which means a chemical moiety with the formula —(CH₂)ₓOC(═S)NRₐR′ₐ and a “N-thiocarbamoyl” group which means a chemical moiety with the formula —(CH₂)ₓNRₐC(═S)OR′ₐ.

The term “thioamide” refers to both, a “C-thioamido” group which means a chemical moiety with the formula —(CH₂)ₓC(═S)NRₐR′ₐ and a “N-thioamido” group which means a chemical moiety with the formula —(CH₂)ₓNRₐC(═S)R′ₐ,

An “urea” group refers to a —(CH₂)ₓNR″ₐC(═O)NRₐR′ₐ.

The term “alkoxy”, as used herein, refers to a chemical moiety of formula —OR_{b}.

The term “alkylthio”, as used herein, refers to a chemical moiety of formula —SR_{b} including the S-oxides thereof.

The term “aryloxy”, as used herein, refers to a chemical moiety of formula —OR_{c}.

The term “arylthio”, as used herein, refers to a chemical moiety of formula —SR_{c} including the S-oxides thereof.

The term “formyl”, as used herein, refers to a chemical moiety of formula —C(═O)H.

The term “oxime ether” as used herein, refers to a chemical moiety of formula —(CH₂)ₓ(Rₐ)C═NORₐa.

By “combined”, when referring to two adjacent “R” groups herein, is meant that the two “R” groups are covalently bonded to each other so as to form a ring system. The ring system may be cycloalkyl, carbocyclic aryl, heteroaryl or heteroalicyclo.

The term “spiroalkyl”, as used herein, refers to an optionally substituted alkyl group where the linkage between the aforementioned alkyl group and a second ring system consists of a single atom common to both groups. The second ring system can be a cycloalkyl or heteroalicyclic group.

x is an integer selected from 0, 1, 2, 3 or 4. One or more hydrogens of a —(CH₂)ₓ group may be replaced by a group selected from hydroxy, halo, cyano, alkoxy, thiol, alkylthio and optionally substituted alkyl and amino. The —(CH₂)ₓ group may also contain double or triple bonds. In such cases, where a double or triple bond exists, the number of hydrogen atoms or substituents is such that the total number of bonds to any one carbon does not exceed 4.

Rₐ, R′ₐ and R″ₐ are independently selected from hydrogen or from an optionally substituted group consisting of alkyl, cycloalkyl, heteroalicyclo and aryl. Rₐ and R′ₐ, Rₐ and R″ₐ and R′ₐ and R″ₐ, when present, may also combine to form a heteroalicyclic ring.

R_{b} is selected from an optionally substituted group consisting of alkyl, cycloalkyl and heteroalicyclo.

R_{c} is an optionally substituted aryl group.

R_{d} is selected from hydrogen or from an optionally substituted group consisting of amino, alkyl, cycloalkyl, heteroalicyclo, carbocyclic aryl, heteroaryl, hydroxy, alkoxy, aryloxy, N-amido, N-thioamido and urea.

Rₑ is selected from the group consisting of hydrogen and hydroxy or from an optionally substituted group consisting of alkyl, alkoxy, aryloxy, cycloalkyl, heteroalicyclo, carbocyclic aryl and heterocyclic aryl, as those terms are defined herein.

R_{f} is selected from the group consisting of hydrogen and cyano or from an optionally substituted group consisting of alkyl, cycloalkyl, heteroalicyclo (bonded through a ring carbon), carbocyclic aryl and heterocyclic aryl (bonded through a ring carbon), as those terms are defined herein.

In order that this invention be more fully understood, the following examples are set forth. These examples are for the purpose of illustrating embodiments of this invention and are not to be construed as limiting the scope of the invention in any way.

The examples which follow are illustrative and, as recognized by one skilled in the art, particular reagents or conditions could be modified as needed for individual compounds. Starting materials used in the scheme below are either commercially available or easily prepared from commercially available materials by those skilled in the art.

### DESCRIPTION OF THE EXAMPLES

### General Experimental Description of Examples

HPLC retention times and mass spectra are recorded according to methods AM1 to AM5. 1H NMR spectra are recorded with either NMR Avance 400 (400, 1330810 MHz) or NMR Avance 500 (500, 1300038 MHz). Microwave heating is performed with either a Personal Chemistry Smith Synthesizer or a CEM Explorer.

Analytical HPLC Method

HPLC: Agilent 1100 Series; MS: 1100 Series LC/MSD Trap (ESI-Mode);
Column: Waters; Part No. 186000594; Xterra MS C18 2.5 μm; 2.1×50 mm column
Solvent: A: H₂O demineralised with 0.1% HCOOH;
Solvent B: MeCN HPLC grade
Detection: MS: scan range: 120-1000 m/z; scan resolution: normal (13000 m/z/min); threshold: 120; Trap: ICC; Target: 1000; Max. Accu. Time: 100 ms; Averages: 10 UV: UV 254 nm (bandwide 1, reference off); UV 230 nm (bandwide 1, reference off); peakwidth>0,05 min (Is); 220-400 nm

Injection: 10 μl standard injection, needle wash

| | | | | |
|---|---|---|---|---|
| Separation: | Flow: 0.6 ml/min | | | |
| | Column temp.: 30 ° C. | | | |
| | Pump 1 (binary): | 0.0 | min | 10% solvent B |
| | | 0.0-2 | min | 10% −> 90% |
| | | | | solvent B |
| | | 4.0-4 | min | 90% solvent B |
| | | 4.5-6.0 | min | 90% −> 10% |
| | | | | solvent B |

Pump 2 (quarternary): 10% solvent B

### Synthesis of Examples

### Examples 1-01 to 1-43

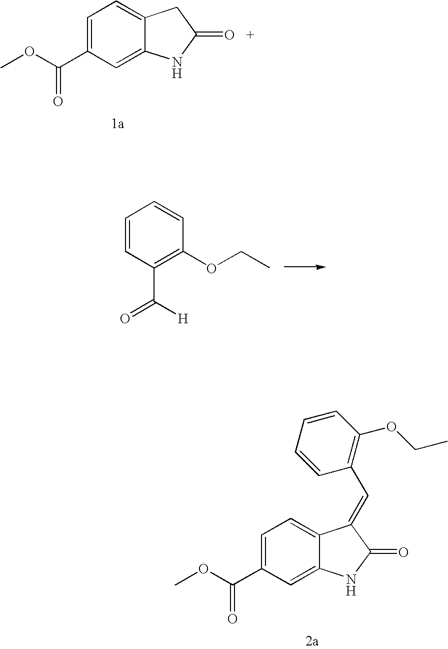

Intermediate 1a is synthesised according to the method described in DE10117204.

Intermediate 1a (9.0 g, 47 mmol), 2-ethoxybenzaldehyde (6.60 mL, 47 mmol) and piperidine (2 mL) are dissolved in methanol (100 mL) and heated under reflux for 2 hours. The solution is allowed to cool to room temperature and the resulting precipitate is collected via filtration and washed with methanol and then ether and dried under reduced pressure to give the desired product 1-01 (13.4 g).

Compounds 1-02 to 1-42 are synthesized from intermediate 1a and the appropriate aldehyde according to the procedure described for 1-01.

### Examples 2-01 to 2-05

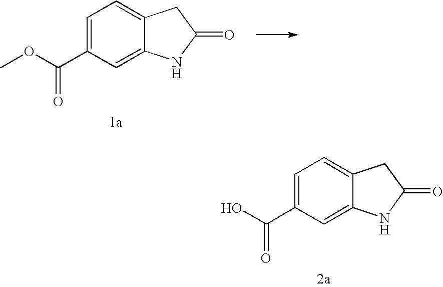

Intermediate 1a (40 g) is dissolved in methanol (400 mL) and 1M NaOH solution (420 mL) is added. The mixture is heated under reflux for 3 hours. The reaction is then neutralized with 1M HCl. The methanol is then removed under reduced pressure and water is added until precipitation commenced. The precipitate is collected via filtration, washed with water and dried under reduced pressure to yield the desired intermediate 2a (37 g)

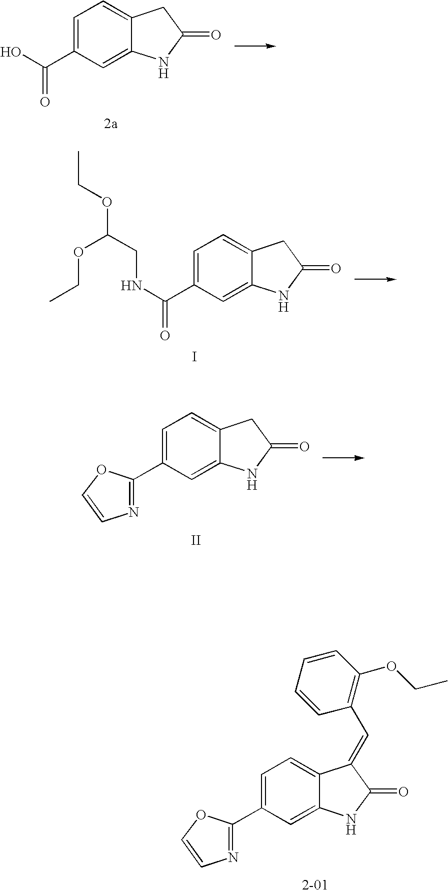

Intermediate 2a (20 g) is dissolved in DMF (150 mL) and carbonyldiimidazole (19 g) is added. The reaction is then heated to 70° C. for 1 hour after which the reaction is cooled to room temperature. Aminoacetaldehyde diethylacetal (16.5 mL) is added and allowed to react at room temperature overnight. The solvent is then removed under reduced pressure and the residue is purified by LC (SiO₂, DCM:MeOH 90:10) to yield the desired compound I (10 g).

Compound I (10 g) is added to polyphosphoric acid (90 g) and heated to 120° C. for 2 hours. The reaction is then cooled to room temperature and poured onto ice. The mixture is neutralized with concentrated ammonia. The resulting precipitate is collected via filtration and then dissolved in DCM:EtOH (9:1). The resulting solution is dried over Na₂SO₄, filtered and the solvent removed in vacuo. The residue is purified by LC (SiO₂, DCM:MeOH 20:1) to yield the desired compound II (0.85 g).

Compounds 2-01 to 2-03 are synthesized from compound II according to the procedure of 1-01.

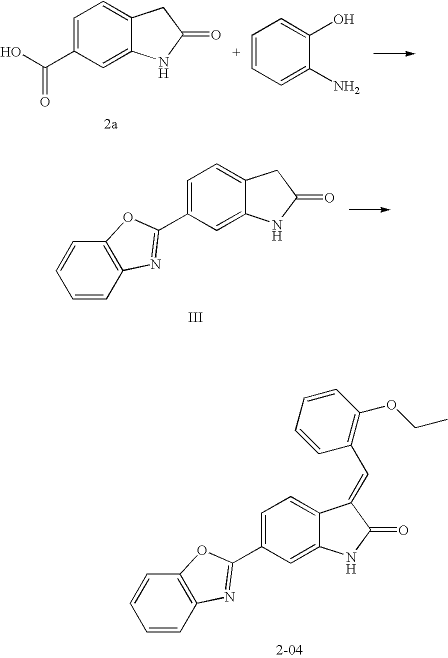

Intermediate 2a (3.3 g) and 2-aminophenol (2 g) are added to polyphosphoric acid (20 g) and heated to 150° C. for 2 hours. The reaction is then cooled to room temperature and then poured onto ice. The mixture is then neutralized with concentrated ammonia. The resulting precipitate is collected via filtration and then dissolved in DCM:EtOH (9:1). The resulting solution is dried over Na₂SO₄, filtered and the solvent is removed in vacuo to yield the desired compound III (660 mg).

Compound 2-04 is synthesized from compound III according to the procedure of 1-01.

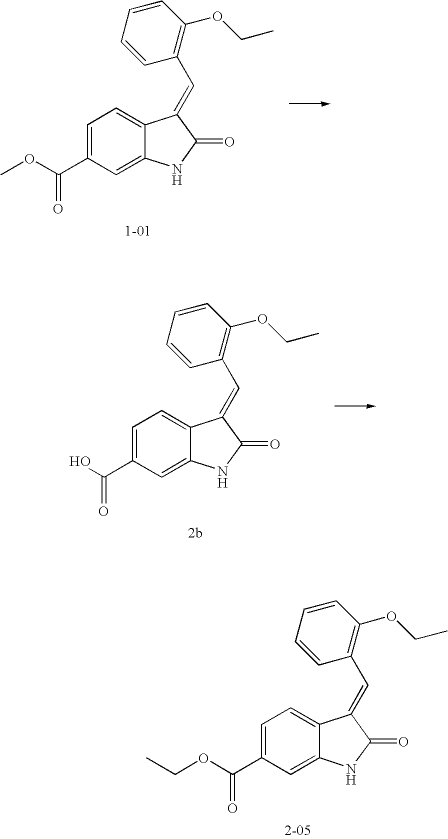

Compound 1-01 (1 g) is dissolved in methanol (7 mL) and 1M NaOH solution (6 mL) is added. The mixture is stirred at room temperature for 48 hours. The resulting precipitate is collected via filtration, washed with methanol and water and dried under reduced pressure over night to yield the desired intermediate 2 b (0.92 g). Compound 2 b (300 mg) is added to ethanol (20 mL) and then cooled to 0° C. Thionyl chloride (420 μl) is added dropwise. The reaction mixture is allowed to warm to room temperature and is then heated to 80 ° C. for 2 hours. The solvent is removed under reduced pressure. Toluene (20 mL) is added and then removed under reduced pressure. The crude material is purified using silica gel and DCM:MeOH (9:1) to yield the desired product 2-05 (280 mg).

Compound 2-06 is synthesized in an analogous manner to 2-05 where propanol is used instead of ethanol.

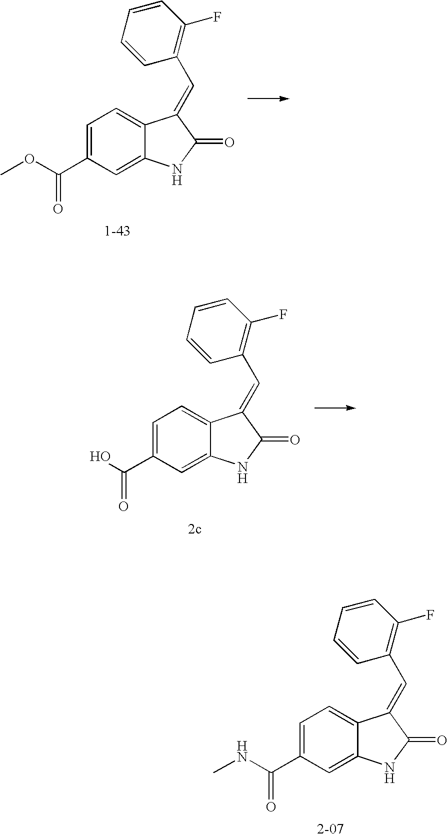

Intermediate 2c is synthesized starting from 1-43 using an analogous procedure to 2b. Intermediate 2c (0.4 g) is dissolved in THF (15 mL) and carbonyldiimidazole (0.24 g) is added. The reaction is then heated to 70° C. for 1 hour after which the reaction is cooled to 0° C. Dimethyl amine (0.1 mL) is added, the reaction is allowed to react further at this temperature for 2 hours and then warmed to room temperature and reacted overnight. The solvent is removed under reduced pressure and partitioned between DCM and water. The organic phase is washed with water, dried over Na₂ SO₄ and the solvent is removed under reduced pressure. The residue is purified by LC (SiO₂, DCM:MeOH 90:10) to yield the desired product 2-07 (58 mg).

Compounds 2-08 and 2-09 are synthesized according to the procedure of 2-07 using the appropriate amine.

### Examples 3-01 to 3-52

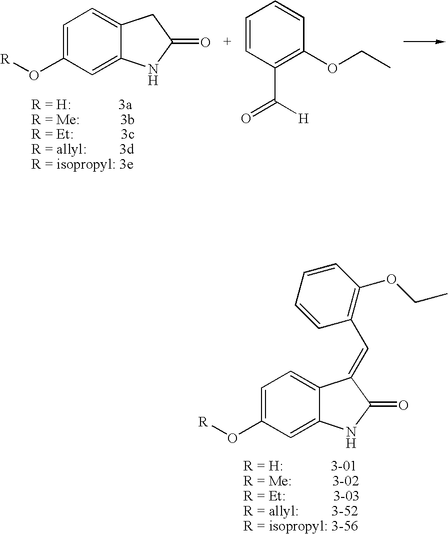

Intermediate 3a is synthesised as described in US6486185. Intermediate 3 b is synthesised as described in WO01064681. Intermediates 3c, 3d and 3e are synthesized according to the procedure of 3b.

Compounds 3-01 to 3-52 are synthesized according to the procedure described for 1-01.

### Examples 4-01 to 4-37

Intermediate 4a is synthesised as described in WO04026829.

Compound 4a (6 g), 2-ethoxybenzaldehyde (4.5 mL) and piperidine (0.6 mL) are added to ethanol (115 mL) and refluxed for 4 hours. The reaction mixture is allowed to cool to room temperature and the resulting precipitate is collected via filtration and dried in vacuo to yield the desired product (4-01, 6.2 g).

The synthesis of example 4-02 is performed using 4a and the appropriate aldehyde as starting material according to the procedure of 4-01.

Compound 4-01 (2 g), pyridine-3-boronic acid (0.8 g), tetrakistriphenylphosphine palladium (0) (0.23 g) and 2M Na₂CO₃ (5.8 mL) are added to dioxane (60 mL) and methanol (10 mL) and heated under reflux for 14 hours. The solvent is removed under reduced pressure and the residue is partitioned between DCM and water. The organic phase is washed with water and dried over Na₂SO₄. The solvent is removed under reduced pressure. The residue is purified by LC (SiO₂, DCM:MeOH 95:5) to yield the desired product 4-03 (0.75 g). Compounds 4-04 to 4-33 are synthesized according to the aforementioned procedure for 4-04 from compound 4-01, the appropriate boronic acid and the appropriate benzaldehyde. Compound 4-03 (120 mg) and methyl iodide (250 mg) are added to chloroform (1.5 mL) and stirred at room temperature overnight. The resulting mixture is filtered and the solid is washed with tert-butylmethyl ether (2×1 mL) to yield the desired compound 4-34 (85 mg). Compound 4-03 (110 mg) and m-chloroperbenzoic acid (397 mg) are added to chloroform (2.5 mL) and methanol (0.5 mL) and stirred at room temperature for 24 hours. The mixture is diluted with DCM (50 mL), washed with saturated Na₂ SO₄ (2×50 mL), diluted Na₂CO₃ (4×50 mL) and brine (1×50 mL), dried over MgSO₄, filtered and concentrated in vacuo. The residue is triturated with tert-butyl methyl ether and the resulting solid collected via filtration to yield the desired product 4-35 (72 mg).

Intermediate 4b is synthesized as described in Giovannini et al., Helvetica Chimica Acto (1948), 31, 1381-91.

Intermediate 4b and 2,5-dimethoxytetrahydrofuran (525 μl) are added to acetic acid (17 mL) and heated to 110° C. for 3 hours. The mixture is then cooled to room temperature, diluted with water and the pH is adjusted to 10 using 2M NaOH. The reaction mixture is extracted using ethyl acetate (4 times). The organic phase is washed with water and saturated brine solution and dried over Na₂SO₄. The solvent is removed under reduced pressure. The residue is dissolved again in ethyl acetate and the resulting solid particles are removed by filtration. The solvent is again removed under reduced pressure to yield the desired intermediate 4c (790 mg).

Intermediate 4c is reacted with 2-allyloxybenzaldehyde according to the procedure described for 1-01 to produce the desired product 4-36.

### Examples 5-01 to 5-02

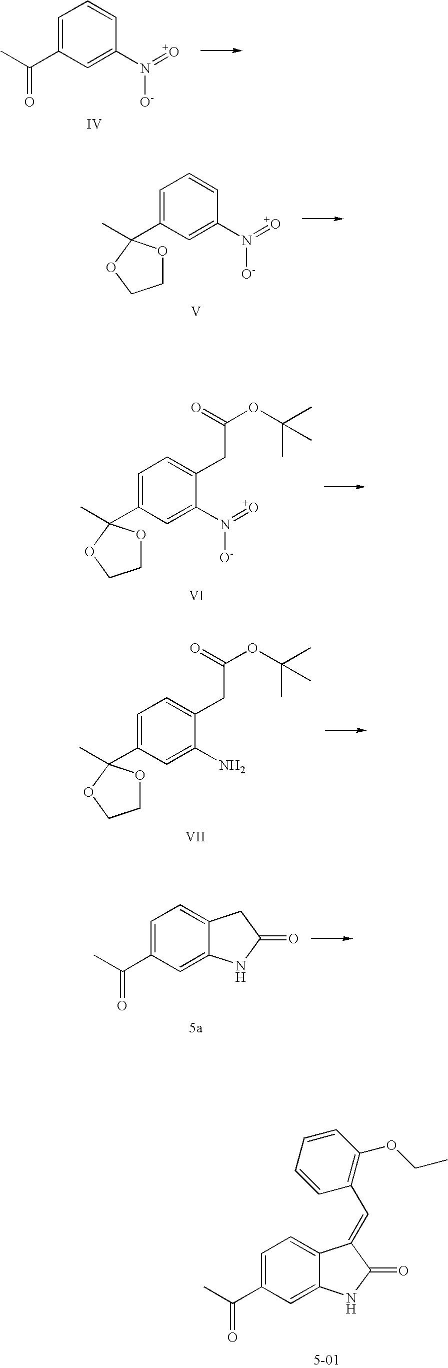

3-Nitroacetophenone (IV) (15 g) and ethyleneglycol (14.3 g) are dissolved in toluene (100 mL). p-Toluenesulfonic acid hydrate (0.35 g) is added. The mixture is heated under reflux for 4 hours. This process is repeated until complete conversion is observed by HPLC/MS. The reaction is cooled to room temperature and partitioned between diethyl ether and 1M NaOH. The organic phase is washed with water, dried over Na₂ SO₄ and the solvent is removed under reduced pressure to give compound V (13.6 g).

Potassium tert-butylate (19 g) is added to DMF (130 mL) and cooled to −5° C. Compound V and tert-butyl chloroacetate (10.8 g) are dissolved in DMF (40 mL) and added dropwise to the above solution over 20 minutes. The reaction is then allowed to warm to room temperature and reacted further until no starting material is observed by HPLC/MS. The reaction is poured onto ice/HCl and extracted with DCM. The organic phase is washed with water, dried over Na₂ SO₄ and the solvent is removed under reduced pressure. The crude material is purified using silica gel and DCM:PE (4:1) to yield the desired compound VI (3.1 g).

Compound VI (3.10 g) is dissolved in methanol (50 mL) and Rancy-Nickel (1.6 g) is added. The reaction mixture is placed under a 50 psi atmosphere hydrogen and is stirred at room temperature for 5 hours. The mixture is then filtered, the filtrate collected and the solvent is removed under reduced pressure to yield the desired product VII (2.6 g)

Compound VII (2.5 g) is dissolved in 1M HCl (40 mL) and methanol (10 mL) and heated under reflux for 1.5 hours. The reaction mixture is then cooled to room temperature and extracted with DCM. The organic phase is washed with water, dried over Na₂SO₄ and the solvent is removed under reduced pressure to give the desired intermediate 5a (0.56 g).

Examples 5-01 and 5-02 are synthesized from intermediate 5 a using the procedure described for 1-04.

### Example 6-01

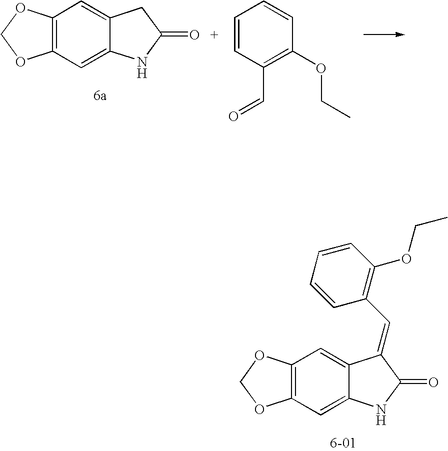

The synthesis of intermediate 7a is described in EP156603

Example 6-01 is synthesized from intermediate 6a according to the procedure of 1-01.

### Example 7-01

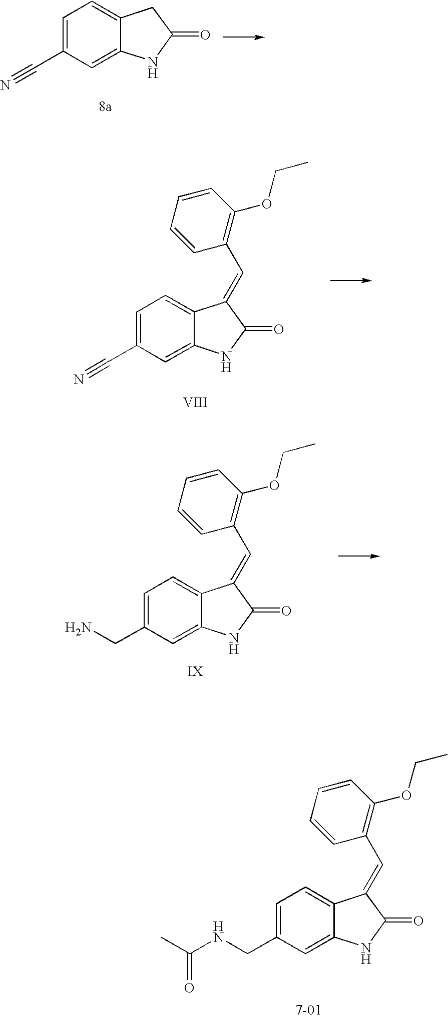

The synthesis of intermediate 8a is described in WO04009546.

Compound VIII is synthesized from intermediate 8a according to the procedure described for 1-01.

Compound VIII (1.0 g) is added to methanol (100 mL) and methanol/NH₄OH (50:%0, 100 mL) followed by Raney-Nickel (0.70 g). The reaction mixture is placed under a 3 bar atmosphere hydrogen and is stirred at room temperature for 6 hours. The mixture is filtered and the filtrate collected. The solvent is removed under reduced pressure The residue is purified by LC (SiO2, DCM:MeOH 9:1) to yield the desired compound IX (0.38 g). Compound IX (150 mg), acetyl chloride (0.04 mL) and triethyl amine (0.08 mL) are added to DCM (5 mL) and stirred at room temperature for 2 hours. The reaction mixture is partitioned between DCM and water. The organic phase is washed with water, dried over Na₂ SO₄ and the solvent removed under reduced pressure to yield the desired product 7-01 (170 mg).

Table of Intermediates

| Number | Structure |
|---|---|
| 1a | 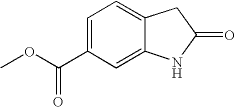 |
| 1b | 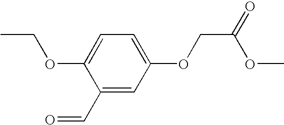 |
| 1c | 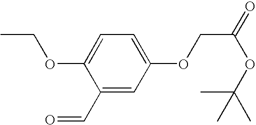 |
| 1d | 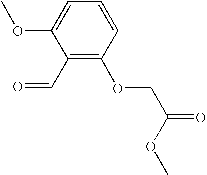 |
| 2a | 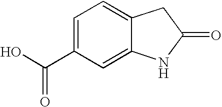 |
| 2b | 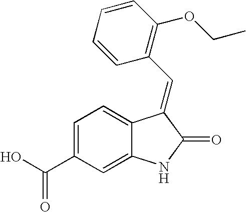 |
| 2c | 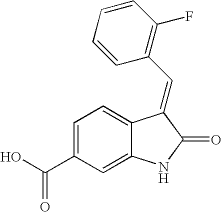 |
| 3a | 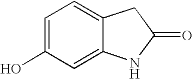 |
| 3b | 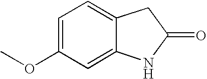 |
| 3c | 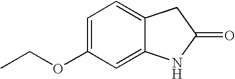 |
| 3d | 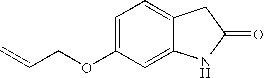 |
| 3e | 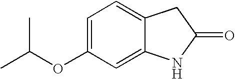 |
| 3f | 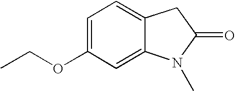 |
| 3g | 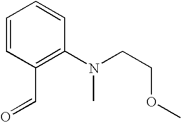 |
| 3h | 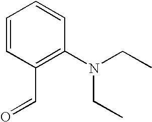 |
| 3i | 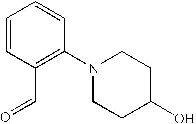 |
| 3j | 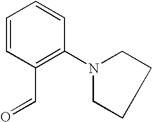 |
| 3k | 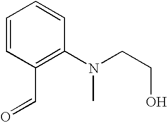 |
| 3l | 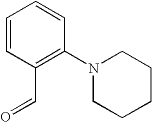 |
| 4a | 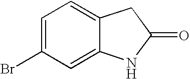 |
| 4b | 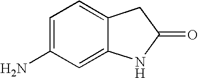 |
| 4c | 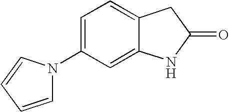 |
| 5a | 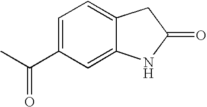 |
| 6a | 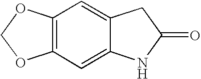 |
| 7a | 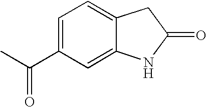 |

Examples 1-01 to 1-42

| | | HPLC/MS | HPLC/MS | |
|---|---|---|---|---|
| Number | Structure | RT #1 | RT #2 | Mass Spectra |
| 1-01 | 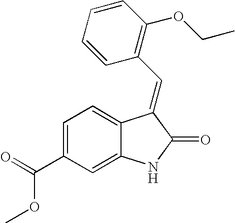 | 3.98 | 4.11 | 324 [M + H]¹⁺ |
| 1-02 | 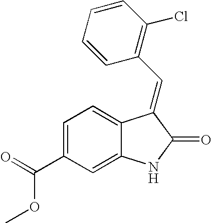 | 3.94 | 4.08 | 314 [M + H]¹⁺ |
| 1-03 | 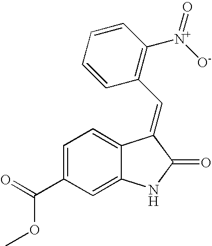 | 3.61 | 3.68 | 325 [M + H]¹⁺ |
| 1-04 | 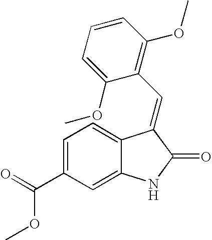 | 3.73 | 3.77 | 340 [M + H]¹⁺ |
| 1-05 | 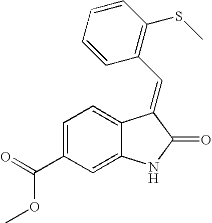 | 3.94 | 4.04 | 326 [M + H]¹⁺ |
| 1-06 | 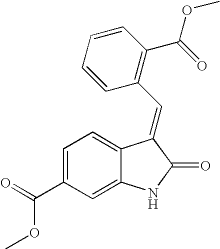 | 3.71 | | not detected |
| 1-07 | 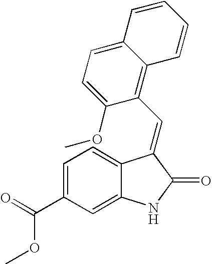 | 4.02 | 4.08 | 360 [M + H]¹⁺ |
| 1-08 | 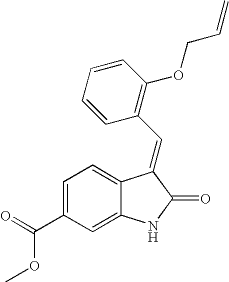 | 4.04 | 4.16 | 336 [M + H]¹⁺ |
| 1-09 | 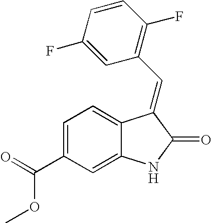 | 3.81 | 4.03 | 316 [M + H]¹⁺ |
| 1-10 | 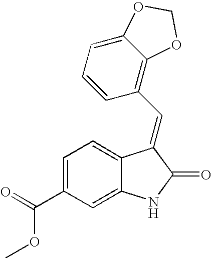 | 3.75 | 3.86 | 324 [M + H]¹⁺ |
| 1-11 | 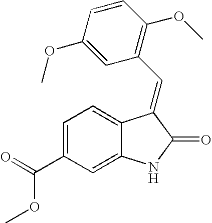 | 3.77 | 3.95 | 340 [M + H]¹⁺ |
| 1-12 | 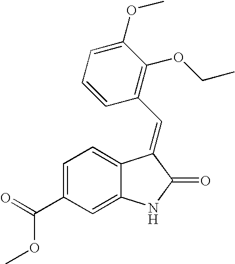 | 3.89 | 4.02 | 354 [M + H]¹⁺ |
| 1-13 | 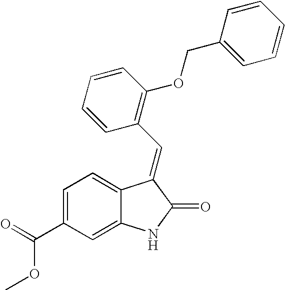 | 4.26 | 4.38 | 386 [M + H]¹⁺ |
| 1-14 | 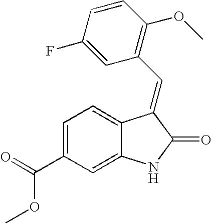 | 3.82 | 3.99 | 328 [M + H]¹⁺ |
| 1-15 | 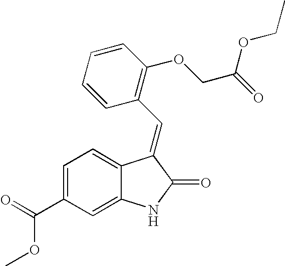 | 3.86 | 3.94 | 382 [M + H]¹⁺ |
| 1-16 | 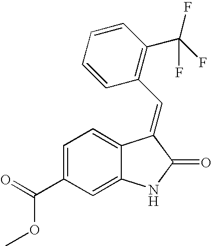 | 3.97 | 4.06 | 348 [M + H]¹⁺ |
| 1-17 | 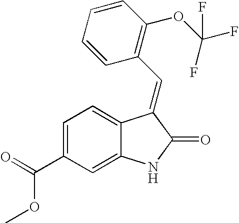 | 4.05 | 4.20 | 363 [M + H]¹⁺ |
| 1-18 | 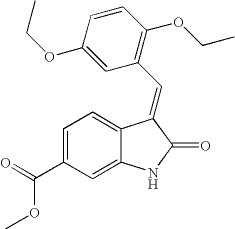 | 4.15 | 4.38 | 368 [M + H]¹⁺ |
| 1-19 | 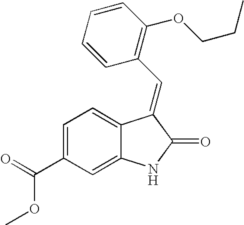 | 4.19 | 4.35 | 338 [M + H]¹⁺ |
| 1-20 | 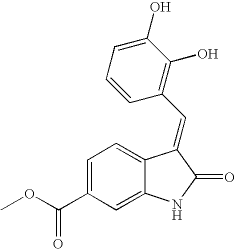 | 3.24 | | 312 [M + H]¹⁺ |
| 1-21 | 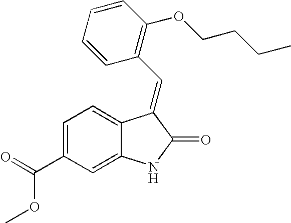 | 4.41 | 4.57 | 352 [M + H]¹⁺ |
| 1-22 | 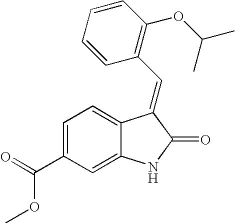 | 4.13 | 4.27 | 338 [M + H]¹⁺ |
| 1-23 | 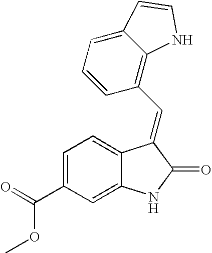 | 3.87 | | 319 [M + H]¹⁺ |
| 1-24 | 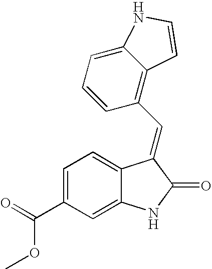 | 3.56 | 3.69 | 319 [M + H]¹⁺ |
| 1-25 | 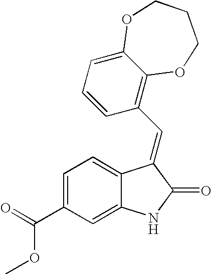 | 3.83 | 3.88 | 352 [M + H]¹⁺ |
| 1-26 | 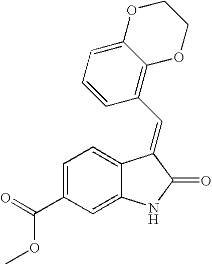 | 3.78 | 3.82 | 338 [M + H]¹⁺ |
| 1-27 | 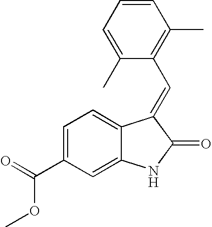 | 4.04 | | 308 [M + H]¹⁺ |
| 1-28 | 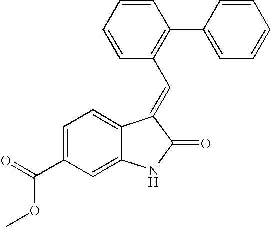 | 4.31 | | 356 [M + H]¹⁺ |
| 1-29 | 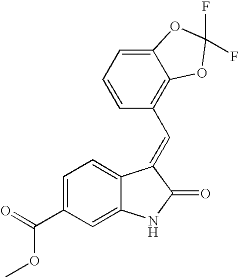 | 4.28 | | 360 [M + H]¹⁺ |
| 1-30 | 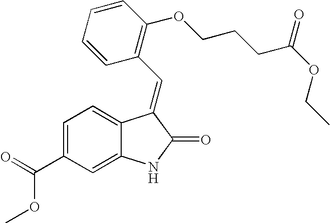 | 4.07 | 4.16 | 410 [M + H]¹⁺ |
| 1-31 | 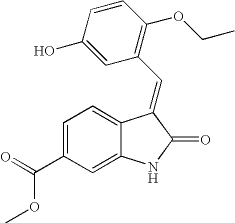 | 3.50 | 3.76 | 340 [M + H]¹⁺ |
| 1-32 | 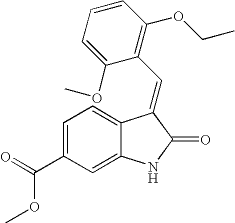 | 3.88 | 3.93 | 354 [M + H]¹⁺ |
| 1-33 | 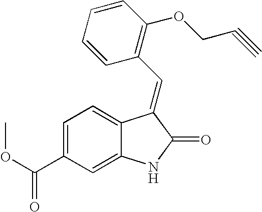 | 3.84 | 3.91 | 334 [M + H]¹⁺ |
| 1-34 | 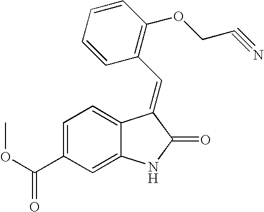 | 3.62 | 3.68 | 335 [M + H]¹⁺ |
| 1-35 | 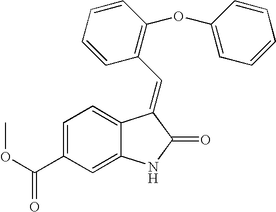 | 4.28 | 4.40 | 372 [M + H]¹⁺ |
| 1-36 | 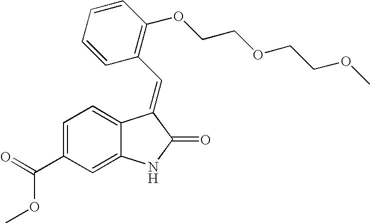 | 3.71 | 3.77 | 398 [M + H]¹⁺ |
| 1-37 | 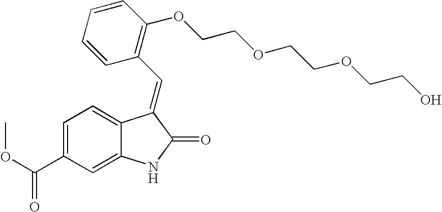 | 3.46 | | 428 [M + H]¹⁺ |
| 1-38 | 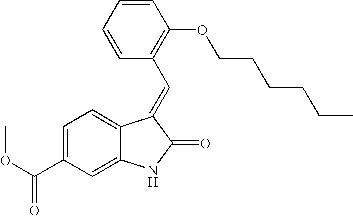 | 4.76 | 4.95 | 380 [M + H]¹⁺ |
| 1-39 | 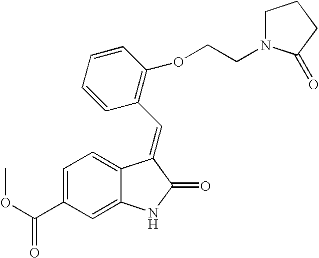 | 3.48 | | 407 [M + H]¹⁺ |
| 1-40 | 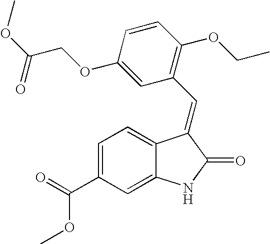 | 3.79 | 4.07 | 412 [M + H]¹⁺ |
| 1-41 | 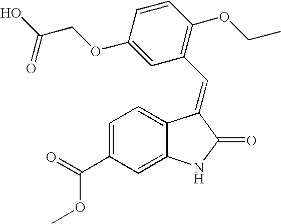 | 3.47 | 3.77 | 398 [M + H]¹⁺ |
| 1-42 | 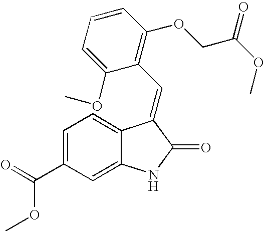 | 3.64 | 3.66 | 398 [M + H]¹⁺ |

Examples 2-01 to 2-09

| | | HPLC/MS | HPLC/MS | |
|---|---|---|---|---|
| Number | Structure | RT #1 | RT #2 | Mass Spectra |
| 2-01 | 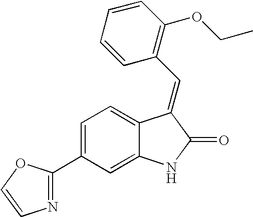 | 3.91 | 4.03 | 333 [M + H]¹⁺ |
| 2-02 | 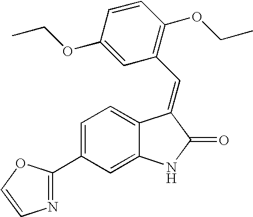 | 4.07 | 4.29 | 377 [M + H]¹⁺ |
| 2-03 | 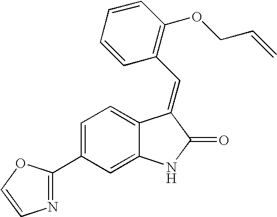 | 3.96 | 4.08 | 345 [M + H]¹⁺ |
| 2-04 | 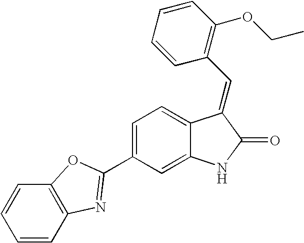 | 4.49 | 4.62 | 383 [M + H]¹⁺ |
| 2-05 | 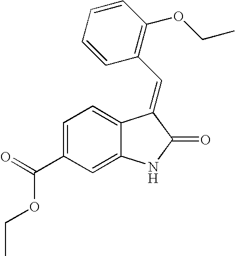 | 4.18 | 4.32 | 338 [M + H]¹⁺ |
| 2-06 | 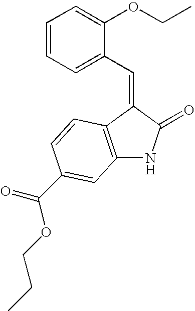 | 4.40 | 4.53 | 352 [M + H]¹⁺ |
| 2-07 | 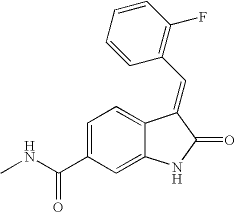 | 3.22 | 3.30 | 297 [M + H]¹⁺ |
| 2-08 | 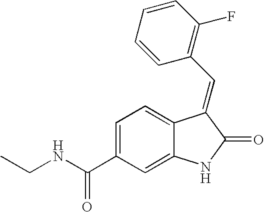 | 3.36 | 3.44 | 311 [M + H]¹⁺ |
| 2-09 | 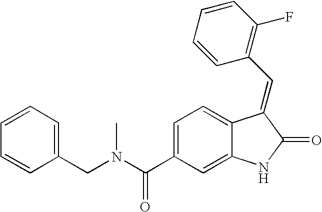 | 3.90 | 4.02 | 387 [M + H]¹⁺ |

Examples 3-01 to 3-56

| | | HPLC/MS | HPLC/MS | |
|---|---|---|---|---|
| Number | Structure | RT #1 | RT #2 | Mass Spectra |
| 3-01 | 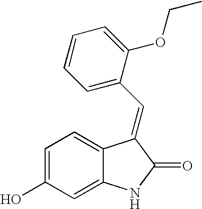 | 3.55 | 3.64 | 282 [M + H]¹⁺ |
| 3-02 | 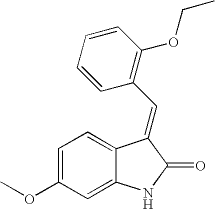 | 3.96 | 4.08 | 296 [M + H]¹⁺ |
| 3-03 | 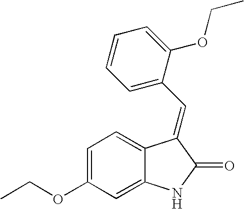 | 4.16 | | 310 [M + H]¹⁺ |
| 3-04 | 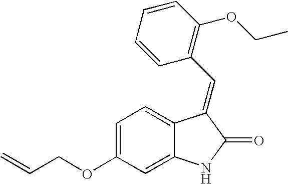 | 4.26 | 4.38 | 322 [M + H]¹⁺ |
| 3-05 | 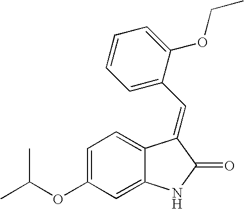 | 4.32 | 4.45 | 324 [M + H]¹⁺ |
| 3-06 | 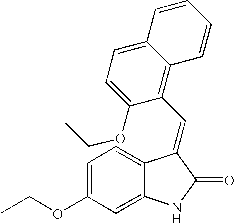 | 4.35 | 4.43 | 360 [M + H]¹⁺ |
| 3-07 | 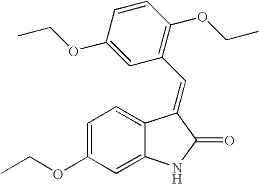 | 4.31 | 4.52 | 354 [M + H]¹⁺ |
| 3-08 | 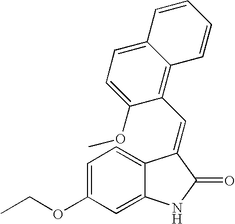 | 4.18 | 4.27 | 346 [M + H]¹⁺ |
| 3-09 | 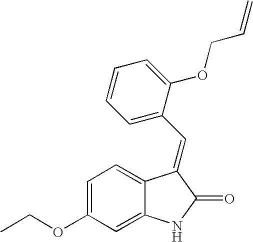 | 4.23 | 4.34 | 322 [M + H]¹⁺ |
| 3-10 | 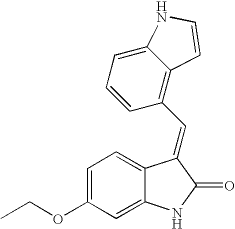 | 3.71 | 3.85 | 305 [M + H]¹⁺ |
| 3-11 | 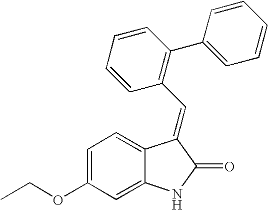 | 4.50 | | 342 [M + H]¹⁺ |
| 3-12 | 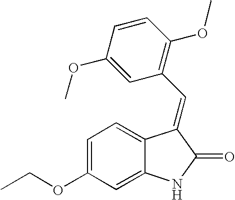 | 3.93 | 4.11 | 325 [M + H]¹⁺ |
| 3-13 | 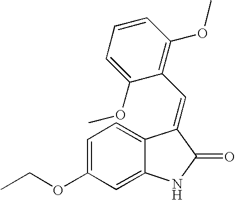 | 3.87 | 3.93 | 326 [M + H]¹⁺ |
| 3-14 | 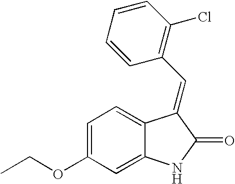 | 4.13 | | 300 [M + H]¹⁺ |
| 3-15 | 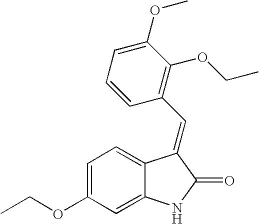 | 4.05 | 4.16 | 340 [M + H]¹⁺ |
| 3-16 | 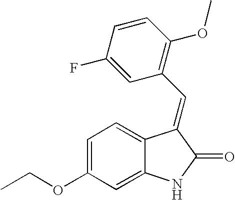 | 4.02 | 4.18 | 314 [M + H]¹⁺ |
| 3-17 | 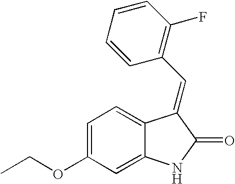 | 4.00 | | 384 [M + H]¹⁺ |
| 3-18 | 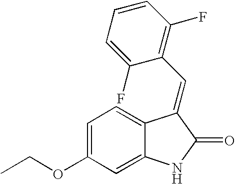 | 3.94 | | 302 [M + H]¹⁺ |
| 3-19 | 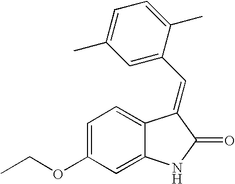 | 4.33 | | 294 [M + H]¹⁺ |
| 3-20 | 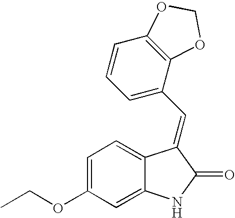 | 3.93 | 4.04 | 310 [M + H]¹⁺ |
| 3-21 | 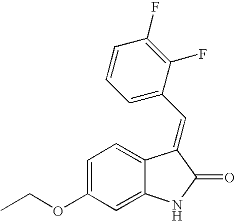 | 4.05 | | 302 [M + H]¹⁺ |
| 3-22 | 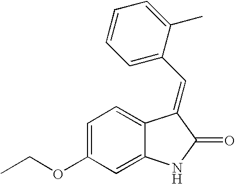 | 4.14 | | 280 [M + H]¹⁺ |
| 3-23 | 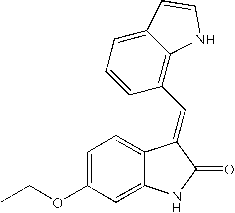 | 4.00 | 4.07 | 305 [M + H]¹⁺ |
| 3-24 | 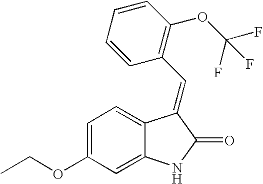 | 4.26 | | 350 [M + H]¹⁺ |
| 3-25 | 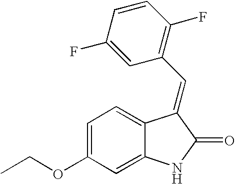 | 4.03 | | 302 [M + H]¹⁺ |
| 3-26 | 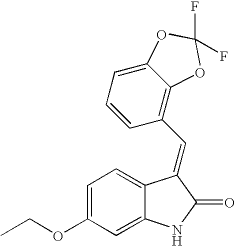 | 4.27 | | 346 [M + H]¹⁺ |
| 3-27 | 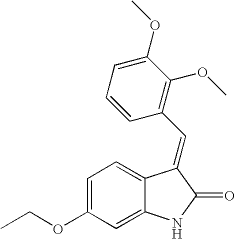 | 3.26 | | 326 [M + H]¹⁺ |
| 3-28 | 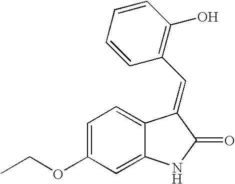 | 3.11 | | 282 [M + H]¹⁺ |
| 3-29 | 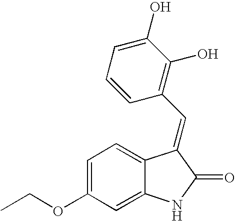 | 4.30 | | 298 [M + H]¹⁺ |
| 3-30 | 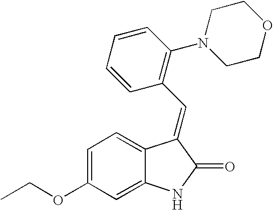 | 4.09 | | 351 [M + H]¹⁺ |
| 3-31 | 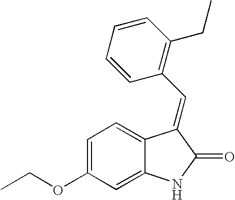 | 4.38 | | 294 [M + H]¹⁺ |
| 3-32 | 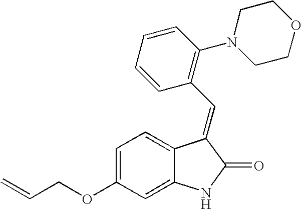 | 4.09 | 4.13 | 363 [M + H]¹⁺ |
| 3-33 | 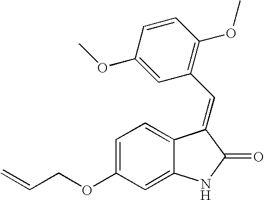 | 4.02 | 4.20 | 338 [M + H]¹⁺ |
| 3-34 | 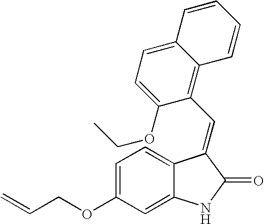 | 4.41 | 4.49 | 372 [M + H]¹⁺ |
| 3-35 | 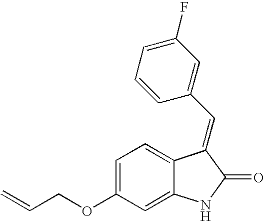 | 4.12 | 4.24 | 296 [M + H]¹⁺ |
| 3-36 | 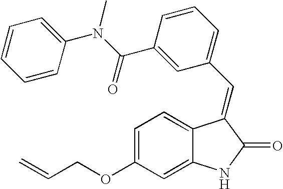 | 3.95 | 4.09 | 411 [M + H]¹⁺ |
| 3-37 | 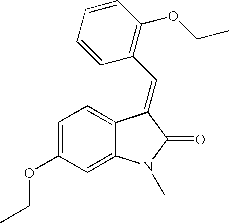 | 3.59 | | 324 [M + H]¹⁺ |
| 3-38 | 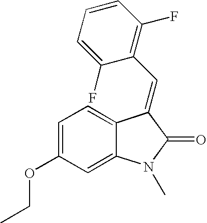 | 3.43 | | 316 [M + H]¹⁺ |
| 3-39 | 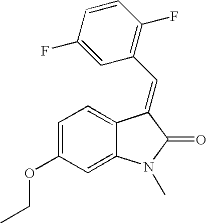 | 3.45 | | 316 [M + H]¹⁺ |
| 3-40 | 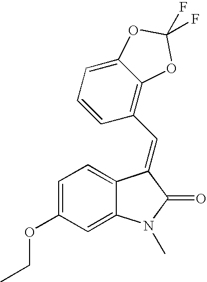 | 3.58 | | 360 [M + H]¹⁺ |
| 3-41 | 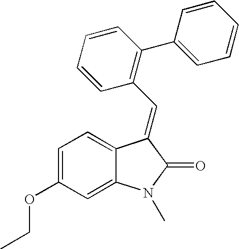 | 3.74 | | 356 [M + H]¹⁺ |
| 3-42 | 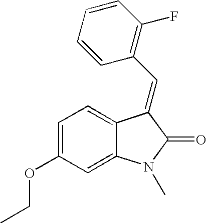 | 3.50 | | 298 [M + H]¹⁺ |
| 3-43 | 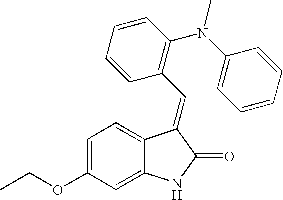 | 4.54 | | 371 [M + H]¹⁺ |
| 3-44 | 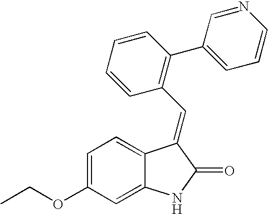 | 3.46 | | 343 [M + H]¹⁺ |
| 3-45 | 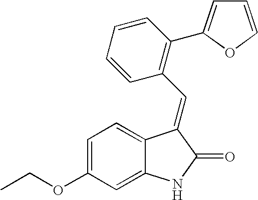 | 4.44 | | 332 [M + H]¹⁺ |
| 3-46 | 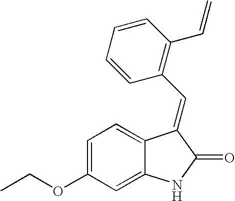 | 4.32 | | 292 [M + H]¹⁺ |
| 3-47 | 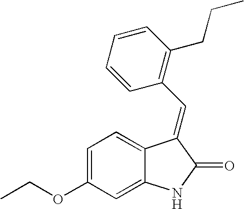 | 4.56 | | 308 [M + H]¹⁺ |
| 3-48 | 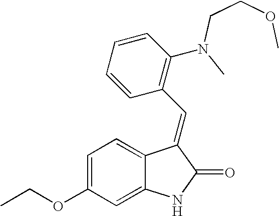 | 4.13 | | 353 [M + H]¹⁺ |
| 3-49 | 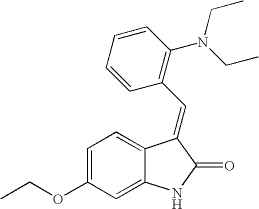 | 3.47 | | 337 [M + H]¹⁺ |
| 3-50 | 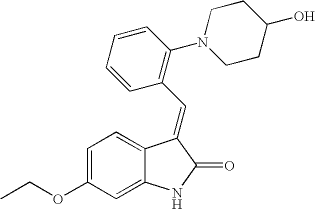 | 3.87 | | 365 [M + H]¹⁺ |
| 3-51 | 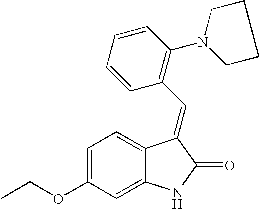 | 4.40 | | 335 [M + H]¹⁺ |
| 3-52 | 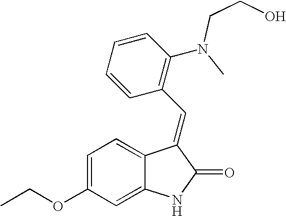 | 3.70 | | 339 [M + H]¹⁺ |
| 3-53 | 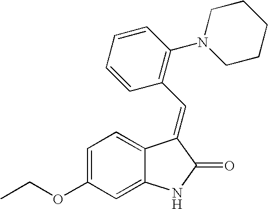 | 4.71 | | 349 [M + H]¹⁺ |

Examples 4-01 to 4-37

| | | HPLC/MS | HPLC/MS | |
|---|---|---|---|---|
| Number | Structure | RT #1 | RT #2 | Mass Spectra |
| 4-01 | 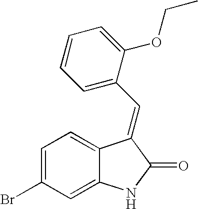 | 4.32 | 4.44 | 344/346 [M + H]¹⁺ |
| 4-02 | 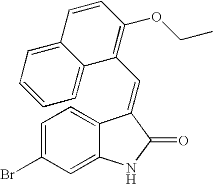 | 4.52 | 4.56 | 394/396 [M + H]¹⁺ |
| 4-03 | 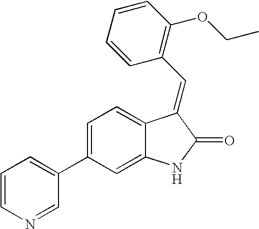 | 3.32 | 3.37 | 343 [M + H]¹⁺ |
| 4-04 | 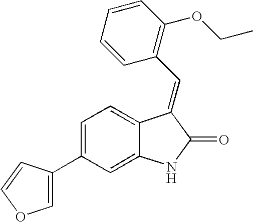 | 4.26 | 4.46 | 332 [M + H]¹⁺ |
| 4-05 | 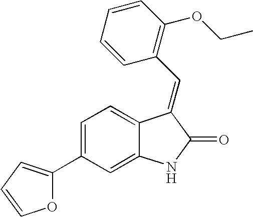 | 4.34 | 4.48 | 332 [M + H]¹⁺ |
| 4-06 | 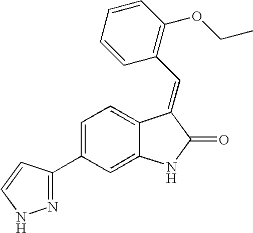 | 3.67 | 3.77 | 332 [M + H]¹⁺ |
| 4-07 | 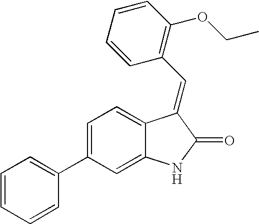 | 4.53 | 4.67 | 342 [M + H]¹⁺ |
| 4-08 | 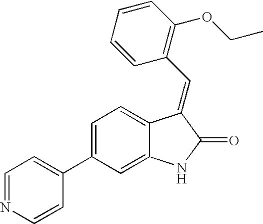 | 3.07 | 3.12 | 343 [M + H]¹⁺ |
| 4-09 | 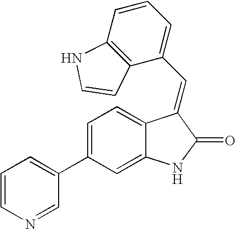 | 2.95 | 3.05 | 338 [M + H]¹⁺ |
| 4-10 | 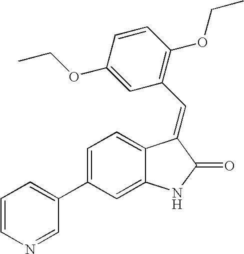 | 3.48 | 3.61 | 387 [M + H]¹⁺ |
| 4-11 | 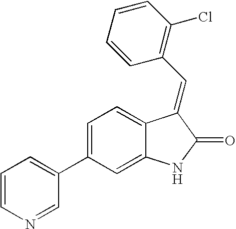 | 3.29 | 3.36 | 333 [M + H]¹⁺ |
| 4-12 | 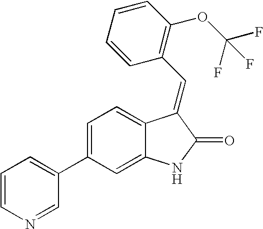 | 3.46 | 3.54 | 383 [M + H]¹⁺ |
| 4-13 | 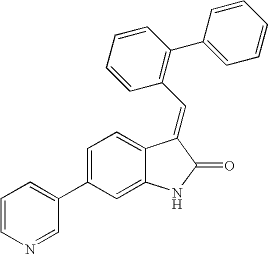 | 3.15 | | 375 [M + H]¹⁺ |
| 4-14 | 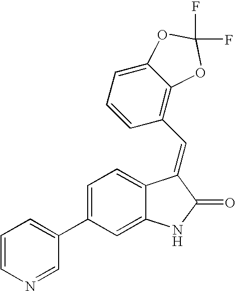 | 3.04 | | 379 [M + H]¹⁺ |
| 4-15 | 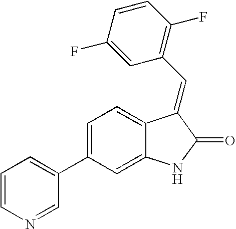 | 2.77 | | 335 [M + H]¹⁺ |
| 4-16 | 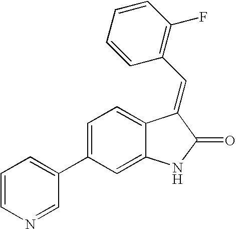 | 2.9 | | 317 [M + H]¹⁺ |
| 4-17 | 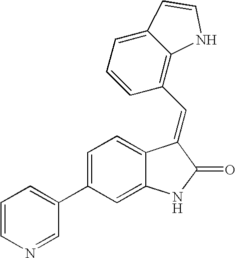 | 2.78 | | 338 [M + H]¹⁺ |
| 4-18 | 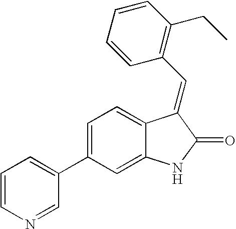 | 3.63 | | 327 [M + H]¹⁺ |
| 4-19 | 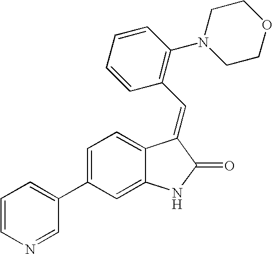 | 3.39 | | 384 [M + H]¹⁺ |
| 4-20 | 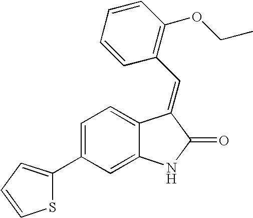 | 4.50 | 4.63 | 348 [M + H]¹⁺ |
| 4-21 | 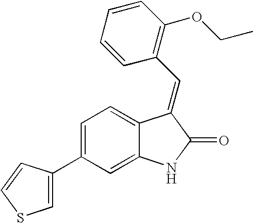 | 4.41 | 4.54 | 348 [M + H]¹⁺ |
| 4-22 | 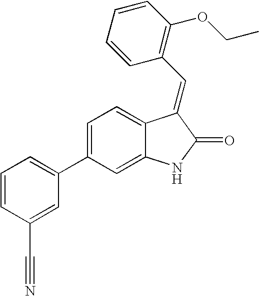 | 4.35 | 4.49 | 367 [M + H]¹⁺ |
| 4-23 | 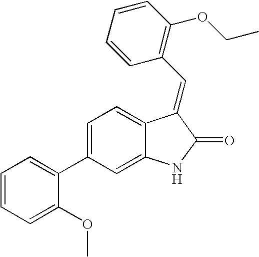 | 4.47 | 4.60 | 372 [M + H]¹⁺ |
| 4-24 | 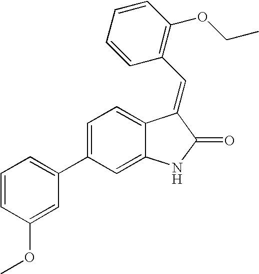 | 4.53 | 4.65 | 372 [M + H]¹⁺ |
| 4-25 | 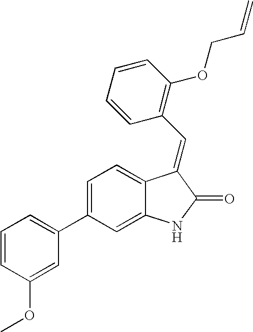 | 4.4 | 4.55 | 384 [M + H]¹⁺ |
| 4-26 | 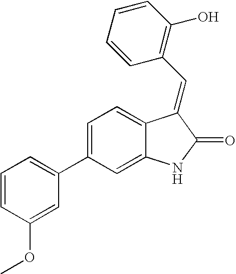 | 2.97 | 3.03 | |
| 4-27 | 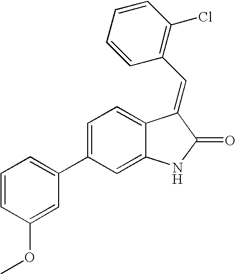 | 4.45 | 4.63 | 362 [M + H]¹⁺ |
| 4-28 | 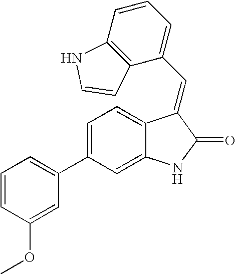 | 4.04 | 4.19 | 367 [M + H]¹⁺ |
| 4-29 | 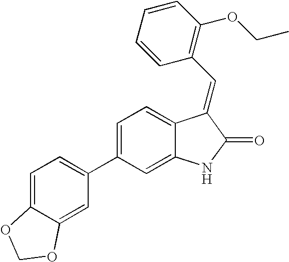 | 4.43 | 4.56 | 386 [M + H]¹⁺ |
| 4-30 | 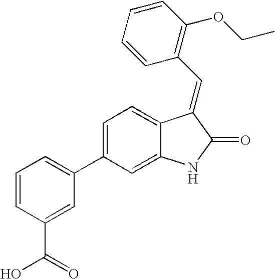 | 4.00 | 4.14 | 386 [M + H]¹⁺ |
| 4-31 | 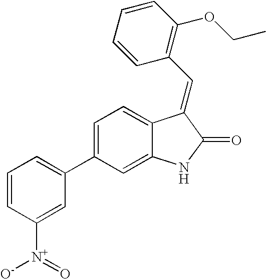 | 4.50 | 4.63 | 387 [M + H]¹⁺ |
| 4-32 | 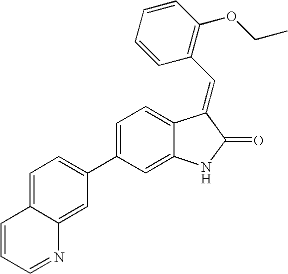 | 4.10 | 4.15 | 393 [M + H]¹⁺ |
| 4-33 | 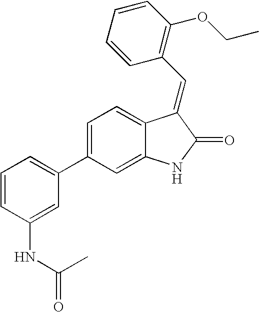 | 3.93 | 4.06 | 399 [M + H]¹⁺ |
| 4-34 | 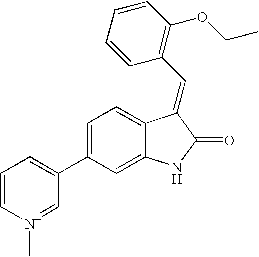 | 2.56 | | 357 [M + H]¹⁺ |
| 4-35 | 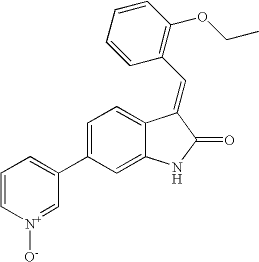 | 2.99 | | 375 [M + H]¹⁺ |
| 4-36 | 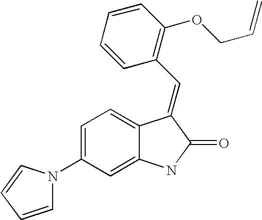 | 4.21 | | 343 [M + H]¹⁺ |
| 4-37 | 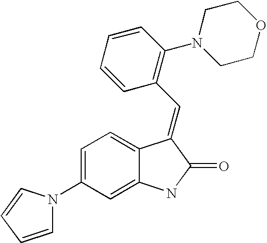 | 4.09 | | 372 [M + H]¹⁺ |

Examples 5-01 and 5-02

| | | HPLC/MS | HPLC/MS | |
|---|---|---|---|---|
| Number | Structure | RT #1 | RT #2 | Mass Spectra |
| 5-01 | 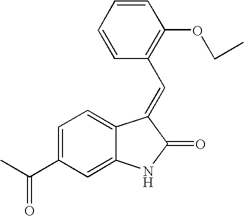 | 3.80 | 3.92 | 308 [M + H]¹⁺ |
| 5-02 | 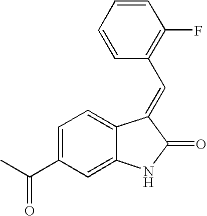 | 3.60 | 3.73 | 282 [M + H]¹⁺ |

Example 6-01

| | | HPLC/MS | HPLC/MS | |
|---|---|---|---|---|
| Number | Structure | RT #1 | RT #2 | Mass Spectra |
| 6-01 | 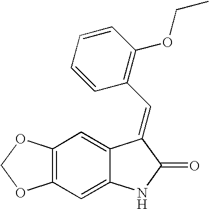 | 3.87 | | 310 [M + H]¹⁺ |

Example 7-01

| | | HPLC/MS | HPLC/MS | |
|---|---|---|---|---|
| Number | Structure | RT #1 | RT #2 | Mass Spectra |
| 7-01 | 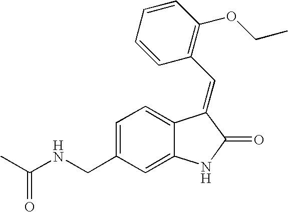 | 3.39 | 3.48 | 337 [M + H]¹⁺ |

Biological Experiments

The compounds of the invention are useful in binding to tubulin and thereby inhibiting the activity of tubulin. In doing so, these compounds are useful in blocking disease processes by binding to tubulin. Accordingly, the compounds of the present invention are useful in treating cancer or other abnormal proliferative diseases. Cancers are classified in two ways: by the type of tissue in which the cancer originates (histological type) and by primary site, or the location in the body where the cancer first developed. The most common sites in which cancer develops include the skin, lungs, female breasts, prostate, colon and rectum, cervix and uterus.

The compounds are thus useful in the treatment of a variety of cancers, including but not limited to the following:
AIDS-related cancer such as Kaposi's sarcoma;
bone related cancer such as Ewing's family of tumors and osteosarcoma;
brain related cancer such as adult brain tumor, childhood brain stem glioma, childhood cerebellar astrocytoma, childhood cerebral astrocytoma/malignant glioma, childhood ependymoma, childhood medulloblastoma, childhood supratentorial primitive neuroectodermal tumors, childhood visual pathway and hypothalamic glioma and other childhood brain tumors;
breast cancer;
digestive/gastrointestinal related cancer such as anal cancer, extrahepatic bile duct cancer, gastrointestinal carcinoid tumor, colon cancer, esophageal cancer, gallbladder cancer, adult primary liver cancer, childhood liver cancer, pancreatic cancer, rectal cancer, small intestine cancer and stomach (gastric) cancer;
endocrine related cancer such as adrenocortical arcinoma, gastrointestinal carcinoid tumor, islet cell carcinoma (endocrine pancreas), parathyroid cancer, pheochromocytoma, pituitary tumor and thyroid cancer;
eye related cancer such as intraocular melanoma, and retinoblastoma;
genitourinary related cancer such as bladder cancer, kidney (renal cell) cancer, penile cancer, prostate cancer, transitional cell renal pelvis and ureter cancer, testicular cancer, urethral cancer, Wilms' tumor and other childhood kidney tumors;
germ cell related cancer such as childhood extracranial germ cell tumor, extragonadal germ cell tumor, ovarian germ cell tumor and testicular cancer;
gynecologic related cancer such as cervical cancer, endometrial cancer, gestational trophoblastic tumor, ovarian epithelial cancer, ovarian germ cell tumor, ovarian low malignant potential tumor, uterine sarcoma, vaginal cancer and vulvar cancer;
head and neck related cancer such as hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, parathyroid cancer and salivary gland cancer;
hematologic/blood related cancer such as leukemias, such as adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; and lymphomas, such as AIDS-related lymphoma, cutaneous T-cell lymphoma, adult Hodgkin's lymphoma, childhood Hodgkin's lymphoma, Hodgkin's lymphoma during pregnancy, mycosis fungoides, adult non-Hodgkin's lymphoma, childhood non-Hodgkin's lymphoma, non-Hodgkin's lymphoma during pregnancy, primary central nervous system lymphoma, Sezary syndrome, cutaneous T-cell lymphoma and Waldenström's macroglobulinemia and other hematologic/blood related cancer such as chronic myeloproliferative disorders, multiple myeloma/plasma cell neoplasm, myelodysplastic syndromes and myelodysplastic/myeloproliferative diseases;
lung related cancer such as non-small cell lung cancer and small cell lung cancer
musculoskeletal related cancer such as Ewing's family of tumors, osteosarcoma, malignant fibrous histiocytoma of bone, childhood rhabdomyosarcoma, adult soft tissue sarcoma, childhood soft tissue sarcoma and uterine sarcoma;
neurologic related cancer such as adult brain tumor, childhood brain tumor, brain stem glioma, cerebellar astrocytoma, cerebral astrocytoma/malignant glioma, ependmoma, medulloblastoma, supratentorial primitive neuroectodermal tumors, visual pathway and hypothalamic glioma and other brain tumors such as neuroblastoma, pituitary tumor and primary central nervous system lymphoma;
respiratory/thoracic related cancer such as non-small cell lung cancer, small cell lung cancer, malignant mesothelioma, thymoma and thymic carcinoma;
skin related cancer such as cutaneous T-cell lymphoma, Kaposi's sarcoma, melanoma, Merkel cell carcinoma and skin cancer

Compounds binding to tubulin may also inhibit angiogenesis and affect abnormal cellular proliferation and can be used to treat certain forms of blindeness related to retinal vascularization, arthritis, especially inflammatory arthritis, multiple sclerosis, restenosis and psoriasis and may induce apoptosis, a physiological cell death process critical for normal development and homeostasis.

The compounds of the invention are also useful for treatment of e.g. follicular lymphomas, carcinomas with p53 mutations, hormone dependent tumor of the breast, prostate and ovary and precancerous lesions such as familial adenomatous polyposis, viral infections, autoimmune diseases such as systemic lupus erythematosus, immune mediated glomerulonephritis, rheumatoid arthritis, psoriasis, inflammatory bowel diseases and autoimmune diabetes mellitus.

The compounds of the invention may be used in combination with other therapies or anticancer agents including surgery, radiotherapy, endocrine therapy, biologic response modifiers, hyperthermia and cryotherapy, agents to attenuate any adverse effect (e.g. antiemetics) and other chemotherapeutic drugs. Such conjoint treatment may be achieved by way of simultaneous, sequential or separate administration of the individual components of the treatment. Chemotherapeutics that may be used in combination with the compounds of the present invention are selected from, but not limited to hormones, hormonal analogues and antihormonals (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortinsone, fluoxymesterone, medroxyprogesterone, octreotide), aromatase inhibitors (e.g. anastrozole, letrozole, liarozole, vorozole, exemestane, atamestane,), LHRH agonists and antagonists (e.g. goserelin acetate, luprolide), inhibitors of growth factor function, (such growth factors include for example platelet derived growth factor and hepatocyte growth factor such inhibitors include growth factor antibodies, growth factor receptor antibodies and tyrosine kinase inhibitors such as gefitinib, imatinib, lapatinib and trastuzumab); antimetabolites (e.g. antifolates like methotrexate, raltitrexed, pyrimidine analogues like 5-fluorouracil capecitabine and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin and idarubicin, mitomycin-C, bleomycin dactinomycin, plicamycin, streptozocin); platinum derivatives (e.g. cisplatin, oxaliplatin, carboplatin); alkylating agents (e.g. estramustine, meclorethamine, melphalan, chlorambucil, busulphan, dacarbazine, cyclophosphamide, ifosfamide, temozolomide, nitrosoureas such as carmustine and lomustine, thiotepa); antimitotic agents (e.g. vinca alkaloids like vinblastine, vindesine, vinorelbine and vincristine; and taxabes like paclitaxel, docetaxel); topoisomerase inhibitors (e.g. epipodophyllotoxins like etoposide and etopophos, teniposide, amsacrine, topotecan, irinotecan, mitoxantrone) and miscellaneous chemotherapeutics such as hydroxyurea, amifostine, anagrelide, clodronate, filgrastin, interferone alpha, leucovorin, rituximab, procarbazine, levamisole, mesna, mitotane, pamidronate and porfimer.

Methods

The in vitro assessment of the biological activity of the inventive compounds is performed as follows:

In vitro Tubulin Polymerization Assay (TPA)

The assay is performed according to Bollag M D et al. (Epothilones, a new class of microtubule-stabilizing agents with a taxol-like mechanism of action. Cancer Research 55: 2325-2333, 1995). Tubulin heterodimers (1.6 mg/ml; 160 μg/assay), from bovine brain (Cytoskeleton), are incubated with test compounds (10 μM final concentration) in PEM (100 mM PIPES, 1 mM EGTA, and 1 mM MgCl₂) buffer (pH 6.6) containing 1 mM GTP in a total volume of 100 μl at 37° C. for 1 h. Samples (80 μl) are then transferred to a 96-well Millipore Multiscreen Durapore hydrophilic 0.22-μm pore size filtration plate. Microtubules are recovered on the filters and are stained with 50 μl of Amido Black solution [0.1% w/v napthol blue black (Sigma), 45% v/v methanol, and 10% v/v acetic acid] for 2 min. Vacuum is applied, and unbound dye is removed by two additions of 200 μl of destain solution (90% v/v methanol, 2% v/v acetic acid). The microtubule bound dye is eluted by incubation with 200 μl of elution solution (25 mM NaOH, 0.05 mM EDTA, and 50% v/v ethanol) for 20 min. Next, 150 μl of elution solution is transferred to a 96-well half area plate, and the absorbance is measured at 600 nm using the Wallac Victor Multilabel counter (Perkin-Elmer/Wallac, Freiburg, Germany). The assay format allows the identification of novel tubulin ligands and gives some indication as to their mechanism of action (e.g. microtubule stabilizer or destabilizer). A result of less than 50% indicates inhibition of tubulin polymerization (destabilizer). A result above 150% indicates induction of tubulin polymerization (stabilizer).

Most of the compounds have values below 50% and are therefore destabilizers.

In vitro Cytotoxicity Assay (MTS)

Cytotoxicity is assessed in HeLa human squamous cell carcinoma by MTS (3-(4, 5-dimethylthiazol-2-yl)-5-(3-carboxymethoxyphenyl)-2-(4-sulfenyl)-2H-tetrazolium, inner salt) assay as reported in T. L. Riss, et. al, “Comparison of MTT, XTT and a novel tetrazolium compound MTS for in vitro proliferation and chemosensitivity assays” Mol. Biol. Cell 3 (Suppl.):184a, 1992

Cells are plated at 2500 cells/well in 96 well microtiter plates and 24 hours later drugs are added and serial diluted (10 μM starting concentration). The cells are incubated at 37° for 4-5 days at which time the tetrazolium dye, MTS at 333 μg/ml (final concentration), in combination with the electron coupling agent phenazine methosulfate at 25 μM (final concentration) is added. The cells are then incubated for 2-3 hours at 37°. The assay is based on the cleavage of the tetrazolium compound MTS to coloured formazan by the “succinate-tetrazolium reductase” mitochondrial enzyme, active only in living (metabolic active) cells. The presence of the electron coupling reagent PMS allows the formation of a stable solution. The amount of dye is quantitated spectrophotometrically at 492 nM. The absorbance is a function of the concentration of converted dye and directly correlates to the number of metabolically active (living) cells in the culture. The results are expressed as an IC50, which is the drug concentration required to inhibit cell proliferation to 50% of that of untreated control cells.

The IC50 values for compounds of this invention fall below 10 μM.

The compounds according to the invention may be administered by oral, transdermal or parenteral route or by inhalation. The compounds according to the invention are present as active ingredients in conventional preparations, e.g. in compositions consisting essentially of an inert pharmaceutical carrier and an effective dose of the active substance, such as for example plain or coated tablets, capsules, lozenges, powders, solutions, suspensions, emulsions, syrups, suppositories, transdermal systems, etc. An effective dose of the compounds according to the invention is between 1 and 100, preferably between 1 and 50, most preferably between 5-30 mg/dose, for oral administration, and between 0.001 and 50, preferably between 0.1 and 10 mg/dose for intravenous or intramuscular administration. For inhalation, solutions containing 0.01 to 1.0, preferably 0.1 to 0.5% of active substance are suitable according to the invention. For inhalation, the use of powders is preferred. It is also possible to use the compounds according to the invention as a solution for infusion, preferably in physiological saline or nutrient salt solution.

The compounds according to the invention may be used on their own or in conjunction with other active substances according to the invention, optionally also in conjunction with other pharmacologically active substances. Suitable preparations include for example tablets, capsules, suppositories, solutions, elixirs, emulsions or dispersible powders. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavoring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates. Solutions for injection and infusion are prepared in the usual way, e.g. with the addition of preservatives such as p-hydroxybenzoates, or stabilizers such as alkali metal salt of ethylenediamine tetra-acetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

A therapeutically effective daily dose is between 1 and 800 mg, preferably 10-300 mg, in adults.

The Examples that follow illustrate the present invention without, however, restricting its scope.

### Examples of Pharmaceutical Formulations

| Tablets | per tablet | |
|---|---|---|
| Active substance | 100 | mg |
| Lactose | 140 | mg |
| Corn starch | 240 | mg |
| Polyvinylpyrrolidone | 15 | mg |
| Magnesium stearate | 5 | mg |
| | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| Tablets | per tablet | |
|---|---|---|
| Active substance | 80 | mg |
| Lactose | 55 | mg |
| Corn starch | 190 | mg |
| Microcrystalline cellulose | 35 | mg |
| Polyvinylpyrrolidone | 15 | mg |
| Sodium-carboxymethyl starch | 23 | mg |
| Magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| Coated tablets | per coated tablet | |
|---|---|---|
| Active substance | 5 | mg |
| Corn starch | 41.5 | mg |
| Lactose | 30 | mg |
| Polyvinylpyrrolidone | 3 | mg |
| Magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| Capsules | per capsule | |
|---|---|---|
| Active substance | 50 | mg |
| Corn starch | 268.5 | mg |
| Magnesium stearate | 1.5 | mg |
| | 320 | mg |

The substance and corn starch are mixed and moistened with water. The moist mass is screened and dried. The dry granules are screened and mixed with magnesium stearate. The finished mixture is packed into size 1 hard gelatine capsules.

Ampoule solution active substance 50 mg sodium chloride 50 mg water for inj. 5 ml

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

Suppositories

| | | |
|---|---|---|
| Active substance | 50 | mg |
| Solid fat | 1650 | mg |
| | 1700 | mg |

The hard fat is melted. At 40° C. the ground active substance is homogeneously dispersed. It is cooled to 38° C. and poured into slightly chilled suppository moulds.

List of Abbreviations

DCM—Dichlormethane
DMF—N,N-Dimethylformamide
EGTA—Ethylene glycol-bis-(2-aminoethyl)-N,N,N′,N′-tetraacetic acid
GTP—Guanidine triphosphate
HPLC—High performance liquid chromatography
LC/MS—Liquid chromatography mass spectrometer
MS—Mass spectrometer
MTS -3-(4, 5-Dimethylthiazol-2-yl)-5-(3-carboxymethoxyphenyl)-2-(4-sulfenyl)-2H-tetrazolium, inner salt
NMR—Nuclear Magnetic Resonance
PIPES—Piperazine-N,N′-bis-(2-ethanesulfonic acid)
PMS—N-Methyldibenzopyrazine methyl sulfate salt
rt—Retention time
RT—Room temperature
THF—Tetrahydrofuran
TPA—Tubulin Polymerisation Assay
UV—Ultraviolet

What is claimed is:

1. A compound of formula (I) 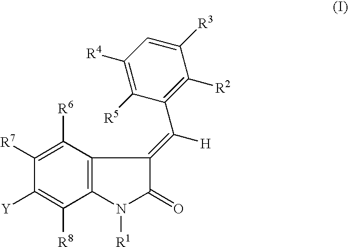
wherein
R¹ is H or methyl; and
R², R³, R⁴ and R⁵ are independently selected from the group consisting of hydrogen, cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ, —(CH₂)ₓNR″ₐC(═O)NRₐR′ₐ and —[(CH₂)ₓO—]_{y}R_{g} or from an optionally substituted group consisting of C₁₋₆alkyl, biaryl, carbocyclic aryl, heteroalicyclo and heteroaryl and
R⁶, R⁷ and R⁸ are independently selected from the group consisting of hydrogen, hydroxy, thiol, halo, cyano, amino, methylamino, dimethylamino, nitro and CF₃ or from an optionally substituted group selected from C₁₋₄alkoxy, C₁₋₄alkylthio, C₁₋₆alkyl, wherein the substituents are selected from the group consisting of halo, hydroxy and oxo; and Y is selected from the group consisting of cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ and —(CH₂)ₓNR″ₐC(═O)NRₐR′ₐ or from an optionally substituted group consisting of C₁₋₆alkyl, biaryl, carbocyclic aryl, heteroalicyclo and heteroaryl; and
R² and R³, R⁴ and R⁵ and R⁷ and Y may also combine to form a cycloalkyl, cycloalkenyl, cycloalkynyl, carbocyclic aryl, heteroalicyclo or heteroaryl ring; and
x is an integer selected from 0, 1, or 2; and
y is an integer selected from 1, 2 or 3; and
Rₐ, R′ₐ and R″ₐ are independently selected from hydrogen or from an optionally substituted group consisting of C₁₋₆alkyl, cycloalkyl, heteroalicyclo and aryl; wherein optionally Rₐ and R′ₐ, Rₐ and R″ₐ and R′ₐ and R″ₐ, may combine to form a heteroalicyclic ring; and
R_{d} is selected from hydrogen or from an optionally substituted group consisting of amino, C₁₋₆alkyl, cycloalkyl, heteroalicyclo, carbocyclic aryl, heteroaryl, C₁₋₄alkoxy, aryloxy, N-amido, N-thioamido and urea; and
Rₑ is selected from the group consisting of hydrogen and hydroxy or from an optionally substituted group consisting of C₁₋₆alkyl, C₁₋₄alkoxy, aryloxy, cycloalkyl, heteroalicyclo, carbocyclic aryl and heterocyclic aryl; and
R_{f} is selected from the group consisting of hydrogen and cyano or from an optionally substituted group consisting of C₁₋₆alkyl, cycloalkyl, heteroalicyclo, carbocyclic aryl and heterocyclic aryl; and
R_{g} is selected from the group consisting of hydrogen and C₁₋₆alkyl,
or a pharmaceutically acceptable salt, solvate or physiologically functional derivative thereof.

2. The compound of formula (I) according to claim 1 wherein
R², R³, R⁴ and R⁵ are independently selected from the group consisting of hydrogen, cyano, hydroxy, halo, nitro, thiol, —(CH₂)ₓC(═O)Rₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓ(Rₐ)C═NR_{d}, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ and —(CH₂)ₓNRₐS(═O)₂R′ₐ or from an optionally substituted group consisting of C₁₋₆alkyl, C₁₋₆alkenyl, C₁₋₆alkynyl, carbocyclic aryl, heteroalicyclo and heteroaryl.

3. The compound of formula (I) according to claim 1 wherein R² is selected from the group consisting of hydrogen, hydroxy, halo, —(CH₂)ₓC(═O)NRₐR′ₐ, —(CH₂)ₓNRₐC(═O)R′ₐ, —(CH₂)ₓNRₐR′ₐ and —(CH₂)ₓORₐ or from an optionally substituted group consisting of carbocyclic aryl, heteroalicyclo and heteroaryl.

4. The compound of formula (I) according to claim 1 wherein
R³ is selected from the group consisting of hydrogen, hydroxy, halo, —(CH₂)ₓC(═O)NRₐR′ₐ, —(CH₂)ₓNRₐC(═O)R′ₐ, —(CH₂)ₓNRₐR′ₐ or —(CH₂)ₓORₐ or from an optionally substituted group consisting of carbocyclic aryl, heteroalicyclo and heteroaryl.

5. The compound of formula (I) according to claim 1 wherein
R² is selected from the group consisting of hydrogen, hydroxy, amino and halo.

6. The compound of formula (I) according to claim 1 wherein
R³ is selected from the group consisting of hydrogen, hydroxy, amino and halo.

7. The compound of formula (I) according to claim 1 wherein
R⁴ is selected from the group consisting of hydrogen, hydroxy, amino and halo.

8. The compound of formula (I) according to claims claim 1 wherein
R⁵ is elected from the group consisting of hydrogen, hydroxy, amino and halo.

9. The compound of formula (I) according to claims claim 1 wherein
Y is selected from the group consisting of hydroxy, halo, thiol, —(CH₂)ₓC(═O)Rₐ, —(CH₂)ₓC(═O)NRₐR′ₐ, —CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓ(Rₐ)C═NR_{d}, (CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, (CH₂)ₓNR″ₐS(═O)₂NRₐR′ₐ, —(CH₂)ₓS(═O)₂NRₐR′ₐ and —(CH₂)ₓNRₐS(═O)₂R′ₐ or from an optionally substituted group consisting of C₁₋₆alkyl, C₁₋₆alkenyl, C₁₋₆alkynyl, carbocyclic aryl, heteroalicyclo and heteroaryl.

10. The compound of formula (I) according to claim 1 wherein
Y is selected from the group consisting of hydroxy, —(CH₂)ₓNRₐR′ₐ, —(CH₂)ₓOC(═O)Rₐ, —(CH₂)ₓC(═O)ORₐ or —(CH₂)ₓORₐ or from an optionally substituted group consisting of carbocyclic aryl, heteroalicyclo and heteroaryl.

11. The compound of formula (I) according to claim 1 wherein
Y is selected from the group consisting of bromo, hydroxy, methoxy, ethoxy, allyloxy, isopropoxy, carboxy, methoxycarbonyl,ethoxycarbonyl, propoxycarbonyl, methylcarbamoyl, ethylcarbamoyl, benzyl-methyl-carbamoyl, oxazol, benzooxazol, furanyl, pyrrolyl, pyrazolyl, thiophenyl, phenyl, cyano-phenyl, methoxy-phenyl, acetylaminophenyl, benzodioxolyl, pyridinyl, methyl-pyridinyl and quinolinyl,

12. The compound of formula (I) according to claim 1 wherein
R⁶, R⁷ and R⁸ are independently selected from the group consisting of hydrogen, hydroxy, halo, cyano, amino, methylamino, dimethylamino, methyl and CF₃.

13. The compound of formula (I) according to claim 1 wherein R¹ is hydrogen.

14. A pharmaceutical composition comprising one or more compounds of formula (I) according to claim 1 and a pharmaceutically acceptable carrier or excipient.

15. A pharmaceutical composition comprising a compound of formula (I) 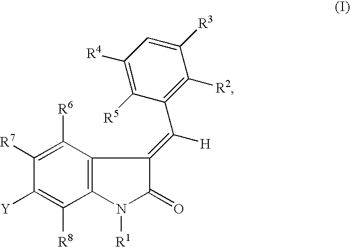
or a salt thereof or a pharmaceutically acceptable derivative thereof, wherein
R¹ is H or methyl; and
R², R³, R⁴ and R⁵ are independently selected from the group consisting of hydrogen, cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓNHC(═NH)NHR_{f}, (CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ, —(CH₂)ₓNR″ₐC(═O)NRₐR′ₐ and —[(CH₂)ₓO—]_{y}R_{g} or from an optionally substituted group consisting of C₁₋₆alkyl, biaryl, carbocyclic aryl, heteroalicyclo and heteroaryl; and
R⁶, R⁷ and R⁸ are independently selected from the group consisting of hydrogen, hydroxy, thiol, halo, cyano, amino, methylamino, dimethylamino, nitro and CF₃ or from an optionally substituted group selected from C₁₋₄alkoxy, C₁₋₄alkylthio, C₁₋₆alkyl, wherein the substituents are selected from the group consisting of halo, hydroxy and oxo;
Y is selected from the group consisting of cyano, isocyanato, isothiocyanato, hydroxy, halo, nitro, thiocyanato, thiol, —(CH₂)ₓC(═O)Rₐ, —(CH₂)ₓC(═NH)NRₐR′ₐ, —(CH₂)ₓC(═O)NRₐR′ₐ, —(CH₂)ₓNRₐC(═O)R′ₐ, —(CH₂)ₓNRₐR′ₐ, —(CH₂)ₓNRₐOR′ₐ, —(CH₂)ₓONRₐR′ₐ, —(CH₂)ₓOC(═O)NRₐR′ₐ, —(CH₂)ₓNRₐC(═O)OR′ₐ, —(CH₂)ₓOC(═O)Rₐ, —(CH₂)ₓC(═O)ORₐ, —(CH₂)ₓORₐ, —(CH₂)ₓNHC(═NH)NHR_{f}, —(CH₂)ₓC(═O)NORₐ, —(CH₂)ₓ(Rₐ)C═NR_{d}, —Si(Rₑ)₃, —(CH₂)ₓS(═O)₂Rₐ, —(CH₂)ₓS(═O)Rₐ, —(CH₂)ₓSRₐ, —(CH₂)ₓS(═O)₂ORₐ, —(CH₂)ₓOS(═O)₂Rₐ, —(CH₂)ₓNR″ₐS(═O)₂NRₐR′ₐ, —(CH₂)ₓS(═O)₂NRₐR′ₐ, —(CH₂)ₓNRₐS(═O)₂R′ₐ, —(CH₂)ₓC(═S)Rₐ, —(CH₂)ₓOC(═S)NRₐR′ₐ, —(CH₂)ₓNRₐC(═S)OR′ₐ, —(CH₂)ₓC(═S)NRₐR′ₐ, —(CH₂)ₓNRₐC(═S)R′ₐ and —(CH₂)ₓNR″ₐC(═O)NRₐR′ₐ or from an optionally substituted group consisting of C₁₋₆alkyl, biaryl, carbocyclic aryl, heteroalicyclo and heteroaryl; and
R² and R³, R⁴ and R⁵ and R⁷ and Y may also combine to form a cycloalkyl, cycloalkenyl, cycloalkynyl, carbocyclic aryl, heteroalicyclo or heteroaryl ring; and
x is an integer selected from 0, 1, or 2; and
y is an integer selected from 1, 2 or 3; and
Rₐ, R′ₐ and R″ₐ are independently selected from hydrogen or from an optionally substituted group consisting of C₁₋₆alkyl, cycloalkyl, heteroalicyclo and aryl; wherein optionally Rₐ and R′ₐ, Rₐ and R″ₐ and R′ₐ and R″ₐ, may combine to form a heteroalicyclic ring; and
R_{d} is selected from hydrogen or from an optionally substituted group consisting of amino, C₁₋₆alkyl, cycloalkyl, heteroalicyclo, carbocyclic aryl, heteroaryl, C₁₋₄alkoxy, aryloxy, N-amido, N-thioamido and urea; and
Rₑ is selected from the group consisting of hydrogen and hydroxy or from an optionally substituted group consisting of C₁₋₆alkyl, C₁₋₄alkoxy, aryloxy, cycloalkyl, heteroalicyclo, carbocyclic aryl and heterocyclic aryl; and
R_{f} is selected from the group consisting of hydrogen and cyano or from an optionally substituted group consisting of C₁₋₆alkyl, cycloalkyl, heteroalicyclo, carbocyclic aryl and heterocyclic aryl; and
R_{g} is selected from the group consisting of hydrogen and C₁₋₆alkyl;
at least one different cytostatic and/or cytotoxic active ingredient or a pharmaceutically acceptable salt, solvate, or physiologically functional derivative thereof, and a pharmaceutically acceptable carrier or excipient.

16. A method for the prevention or treatment of a proliferative disease comprising administration of a therapeutically effective amount of a compound according to claim 1.
